(19) United States
(12) Patent Application Publication
(10) Pub. No.: US 2026/0228893 A1
(43) Pub. Date: Aug. 6, 2026

(54) CONFORMAL PREDICTION TECHNIQUES TO CLASSIFY HISTOPATHOLOGY IMAGES BASED ON POSSIBLE DIAGNOSTIC ENTITIES, WITH CALIBRATED CONFIDENCE LEVELS

(71) Applicant: PathAI, Inc., Boston, MA (US)

(72) Inventors: Jake Conway, Methuen, MA (US); John Shamshoian, Somerville, MA (US); Juhyun Kim, McKinney, TX (US); Santhosh Balasubramanian, San Mateo, CA (US); Shima Nofallah, Brookline, MA (US); Vincent Billaut, Jersey City, NJ (US)

(73) Assignee: PathAI, Inc., Boston, MA (US)

(21) Appl. No.: 19/467,652

(22) Filed: Feb. 2, 2026

Related U.S. Application Data
(60) Provisional application No. 63/753,286, filed on Feb. 3, 2025.
(60) Provisional application No. 63/753,315, filed on Feb. 3, 2025.
(60) Provisional application No. 63/753,336, filed on Feb. 3, 2025.

(51) Int. Cl. G06T 7/00; G06T 3/60; G06V 10/764; G06V 10/82; G06V 20/69

(52) U.S. Cl. CPC G06T 7/0012; G06T 3/60; G06T 2200/24; G06T 2207/10056; G06T 2207/20076; G06T 2207/20081; G06T 2207/20084; G06T 2207/30024; G06T 2207/30088; G06T 2207/30096; G06T 2207/30101; G06V 10/764; G06V 10/82; G06V 20/698; G06V 2201/03

(57) ABSTRACT

In some aspects, a method, a system, or a non-transitory computer-readable storage medium are described for analyzing a histopathology image by obtaining the histopathology image, the image containing histopathology data of one or more tissue samples, processing the histopathology image data using at least one trained machine learning (ML) model to determine a likelihood of one or more diagnostic entities being present in the one or more tissue samples, determining a calibrated probability for each of the determined likelihoods of diagnostic entities being present, generating an output set of one or more diagnostic entities, wherein a size of the output set and the diagnostic entities within the output set are determined based on the calibrated probabilities associated with each diagnostic entity, and causing a user interface to display the output set and indications of the one or more diagnostic entities of the output set.

### CROSS-REFERENCE TO RELATED APPLICATIONS

This application claims priority under 35 U.S.C. § 119 (e) to U.S. Provisional Patent Application Ser. No. 63/753,286, entitled “AUTOMATED ORIENTATION OF A HISTOPATHOLOGY IMAGE TO PREPARE IT FOR OPTIMAL REVIEW BY A HUMAN” filed Feb. 3, 2025, U.S. Provisional Patent Application Ser. No. 63/753,315, entitled “CONFORMAL PREDICTION TECHNIQUES TO CLASSIFY HISTOPATHOLOGY IMAGES BASED ON POSSIBLE DIAGNOSTIC ENTITIES, WITH CALIBRATED CONFIDENCE LEVELS” filed Feb. 3, 2025, and U.S. Provisional Patent Application Ser. No. 63/753,336, entitled “AI-BASED SOFTWARE TOOL FOR ASSISTING THE REVIEW OF DERMATOPATHOLOGY SPECIMENS” filed Feb. 3, 2025, each of which is incorporated by reference herein in its entirety.

### BACKGROUND

Pathology as a medical discipline is instrumental in providing diagnostic and prognostic information to clinicians and patients. In a pathology workflow, biopsies of surgical tissue specimens are collected, stained, and fixed for microscopy. Microscopic analysis of the tissue is used to establish a diagnosis, estimate disease severity, and identify relevant clinical features for treatment.

The practice of pathology is not inherently digital; traditionally, pathology slides are manually examined under a microscope. Microscopy slides are increasingly being digitized in their entirety via slide scanning, generating digital whole slide images (“WSIs” or “slides”). While WSIs provide a wealth of information about a specimen to trained readers such as pathologists, the images themselves are enormous. Each WSI contains up to millions of cells and can be gigapixels in scale, making an exhaustive quantitative manual analysis of WSIs nearly impossible.

### SUMMARY

Some embodiments provide for a method for automated orientation of a histopathology image, the method including using at least one computer hardware processor to perform: obtaining the histopathology image, the histopathology image depicting one or more tissue samples obtained from a biopsy, isolating image data corresponding to a target tissue from the tissues depicted in the histopathology image, identifying a contour of the target tissue using the image data corresponding to the target tissue, identifying an axis associated with the contour of the target tissue, identifying a rotation to be applied to the histopathology image based on the axis and a predetermined preferred orientation of the target tissue, and obtaining a reoriented histopathology image by applying the rotation to the histopathology image.

In some embodiments, isolating the image data corresponding to the target tissue involves: determining tissue types of the one or more tissue samples depicted in the histopathology image, and isolating the target tissue based on the tissue types of the one or more tissue samples.

In some embodiments, identifying the rotation to be applied to the histopathology image involves: determining the predetermined preferred orientation based on the type of the target tissue.

In some embodiments, identifying the rotation to be applied to the histopathology image further involves: determining the rotation to be applied based on an orientation of the target tissue in the histopathology image.

In some embodiments, the predetermined preferred orientation is based on consensus pathologist preference for tissue orientation for the target tissue type.

In some embodiments, the histopathology image depicts tissues obtained from a skin biopsy, and identifying the rotation to be applied to the histopathology image involves identifying an epidermis from the tissues depicted in the histopathology image.

In some embodiments, the tissue samples depicted in the histopathology image have a H&E stain applied, and identifying the epidermis involves identifying the epidermis based on pixel color values of the image data corresponding to the target tissue.

In some embodiments, in the predetermined preferred orientation, the epidermis is depicted facing a top side of the histopathology image.

In some embodiments, identifying the axis associated with the contour of the target tissue involves: determining a shape of the contour, wherein the axis includes one of the major or minor axis of the shape.

In some embodiments, the shape of the contour includes an ellipse bounding the contour.

In some embodiments, the method further includes causing a display device to display the reoriented histopathology.

In some embodiments, the image data corresponding to the target tissue includes multiple target tissue samples, respective rotations to be applied to the histopathology images are identified based on axes and the predetermined preferred orientations of the target tissues for each of the target tissue samples, and obtaining the rotated histopathology image includes applying a weighted average of the respective rotations to the histopathology image.

Some embodiments provide for a system for automated orientation of a histopathology image, the system including: at least one computer hardware processor, and at least one non-transitory computer readable storage medium, storing processor executable instructions, that when executed by the at least one computer hardware processor cause the processor to perform a method including: obtaining the histopathology image, the histopathology image depicting one or more tissue samples obtained from a biopsy, isolating image data corresponding to a target tissue from the tissues depicted in the histopathology image, identifying a contour of the target tissue using the image data corresponding to the target tissue, identifying an axis associated with the contour of the target tissue, identifying a rotation to be applied to the histopathology image based on the axis and a predetermined preferred orientation of the target tissue, and obtaining a reoriented histopathology image by applying the rotation to the histopathology image.

Some embodiments provide for at least one non-transitory computer readable storage medium, storing processor executable instructions, that when executed by at least one computer hardware processor cause the processor to perform a method including: obtaining the histopathology image, the histopathology image depicting one or more tissue samples obtained from a biopsy, isolating image data corresponding to a target tissue from the tissues depicted in the histopathology image, identifying a contour of the target tissue using the image data corresponding to the target tissue, identifying an axis associated with the contour of the target tissue, identifying a rotation to be applied to the histopathology image based on the axis and a predetermined preferred orientation of the target tissue, and obtaining a reoriented histopathology image by applying the rotation to the histopathology image.

Some embodiments provide for a method for analyzing a histopathology image, the method including: using at least one computer hardware processor to perform: obtaining the histopathology image, the image containing histopathology data of one or more tissue samples, processing the histopathology image data using at least one trained machine learning (ML) model to determine a likelihood of one or more diagnostic entities being present in the one or more tissue samples, determining a calibrated probability for each of the determined likelihoods of diagnostic entities being present, generating an output set of one or more diagnostic entities, wherein a size of the output set and the diagnostic entities within the output set are determined based on the calibrated probabilities associated with each diagnostic entity, and causing a user interface to display the output set and indications of the one or more diagnostic entities of the output set.

In some embodiments, determining a calibrated probability for each of the determined likelihoods of diagnostic entities being present includes applying one of: temperature scaling, vector scaling matrix scaling, or Dirichlet calibration to the likelihoods output by the ML model to obtain the calibrated probabilities.

In some embodiments, generating the output set includes applying a conformal prediction technique to the calibrated probabilities.

In some embodiments, applying the conformal prediction technique includes applying a score function to each of the calibrated probabilities to determine respective scores, and comparing the scores to a threshold score associated with a quantile determined for the ML model, when the score associated with a diagnostic entity exceeds the threshold score, including the diagnostic entity in the output set.

In some embodiments, the conformal prediction technique is an adaptive prediction set technique.

In some embodiments, applying the score function includes: sorting the calibrated probabilities from most to least likely, and greedily summing the probabilities.

In some embodiments, the output set is the set of diagnostic entities for which the greedily summed probabilities exceeds the threshold score.

In some embodiments, the output set is the set of entities satisfying the conformal prediction technique and/or having a size less than or equal to a maximum set size.

In some embodiments, the maximum set size is three diagnostic entities.

In some embodiments, the at least one trained ML model includes: a pre-trained foundational model backbone, and an adaptation head trained to determine the likelihood of the one or more diagnostic entities being present in one or more tissue samples.

In some embodiments, the indications of one or more of the diagnostic entities include image data labeled with one or more fields of interest (FOIs) corresponding to a diagnostic entity within the output set.

In some embodiments, the indications of the one or more diagnostic entities include image data with one or more visual overlays, each overlay indicating the likelihood of one or more diagnostic entities being present.

In some embodiments, the one or more diagnostic entities include: benign nevus, basal cell carcinoma, squamous cell carcinoma, seborrheic keratosis, actinic keratosis, dermatitis, dysplastic nevus, squamous cell carcinoma in situ, verruca vulgaris, benign non-melanocytic lesion, scar, cyst, lichenoid keratosis, melanoma in situ, vascular lesion, invasive melanoma, and/or normal skin.

Some embodiments provide for a system for processing of a histopathology image, the system including: at least one computer hardware processor, and at least one non-transitory computer readable storage medium, storing processor executable instructions, that when executed by the at least one computer hardware processor cause the processor to perform a method including: obtaining the histopathology image, the image containing histopathology data of one or more tissue samples, processing the histopathology image data using at least one trained machine learning (ML) model to determine a likelihood of one or more diagnostic entities being present in the one or more tissue samples, determining a calibrated probability for each of the determined likelihoods of diagnostic entities being present, generating an output set of one or more diagnostic entities, wherein a size of the output set and the diagnostic entities within the output set are determined based on the calibrated probabilities associated with each diagnostic entity, and causing a user interface to display the output set and indications of the one or more diagnostic entities of the output set.

Some embodiments provide for at least one non-transitory computer readable storage medium, storing processor executable instructions, that when executed by at least one computer hardware processor cause the processor to perform a method including: obtaining the histopathology image, the image containing histopathology data of one or more tissue samples, processing the histopathology image data using at least one trained machine learning (ML) model to determine a likelihood of one or more diagnostic entities being present in the one or more tissue samples, determining a calibrated probability for each of the determined likelihoods of diagnostic entities being present, generating an output set of one or more diagnostic entities, wherein a size of the output set and the diagnostic entities within the output set are determined based on the calibrated probabilities associated with each diagnostic entity, and causing a user interface to display the output set and indications of the one or more diagnostic entities of the output set.

Some embodiments provide for a method for preparing a histopathology image for review, the method including: using at least one computer hardware processor to perform: obtaining the histopathology image, the image containing histopathology data of one or more tissue samples, processing the histopathology image data using at least one trained machine learning model to determine a likelihood of each of a plurality of diagnostic entities being present in the one or more tissue samples, determining an output set of diagnostic entities by applying a conformal prediction technique to the determined likelihoods, wherein the size of the output set determined by the conformal prediction technique is determined based on the determined likelihoods, generating an output image by applying indications of a presence of the output set of diagnostic entities to the histopathology image, and causing a user interface to display the output image.

In some embodiments, applying the indications of the presence of one or more of the diagnostic entities includes applying one or more labeled fields of interest (FOIs) on the output image.

In some embodiments, applying the one or more labeled fields of interest on the output image includes: applying at least one trained ML model to the histopathology image to identify features associated with one or more diagnostic entities and areas of background within the histopathology image, identifying, based on the features associated with the one or more diagnostic entities, portions of the image having areas of features associated with the one or more diagnostic entities greater than a threshold area, and determining the FOIs from the identified portions of the image based on the areas of the portions of the image.

In some embodiments, the FOIs include enlarged portions of the histopathology image.

In some embodiments, the FOIs include portions of the histopathology image bound by a border.

In some embodiments, the indications of the one or more diagnostic entities include image data with one or more visual overlays, each overlay indicating the likelihood of one or more diagnostic entities being present.

In some embodiments, the method further includes determining one or more recommended histopathology stains based on the determined likelihoods of each the plurality of diagnostic entities, and causing the user interface to display the one or more recommended histopathology stains.

In some embodiments, generating the output image includes determining a rotation to be applied to the output image, and orienting the output image based on the rotation to be applied.

In some embodiments, determining the rotation to be applied includes: isolating a target tissue from the tissues depicted in the histopathology image, wherein the histopathology image depicts tissues obtained from a skin biopsy, and wherein isolating the target tissue includes isolating an epidermis from the tissues depicted in the histopathology image, determining an axis of the target tissue based on the epidermis, wherein the rotation to be applied is determined based on the axis and a desired orientation of the target tissue.

In some embodiments, the method further includes when the one or more diagnostic entities includes a cancer, determining whether the histopathology image contains margin ink, when the histopathology image contains margin ink, performing a tumor margin assessment, and causing the user interface to display a representation of the tumor margin assessment.

In some embodiments, performing the tumor margin assessment includes: processing the histopathology image using one or more ML models to identify portions of the histopathology image corresponding to margin ink, usable tissue, and cancer, based on the usable tissue, identifying pixels of the histopathology image corresponding to one or more margins for tissue sections of the one or more tissue samples in the histopathology image, and determining a margin distance for each tissue section as the minimum distance between cancer pixels contained in the tissue section and the margin of the tissue section.

Some embodiments provide for a system for preparing a histopathology image for review, the system including at least one computer hardware processor, and at least one non-transitory computer readable storage medium, storing processor executable instructions, that when executed by the at least one computer hardware processor cause the processor to perform a method including: obtaining the histopathology image, the image containing histopathology data of one or more tissue samples, processing the histopathology image data using at least one trained machine learning model to determine a likelihood of each of a plurality of diagnostic entities being present in the one or more tissue samples, determining an output set of diagnostic entities by applying a conformal prediction technique to the determined likelihoods, wherein the size of the output set determined by the conformal prediction technique is determined based on the determined likelihoods, generating an output image by applying indications of a presence of the output set of diagnostic entities to the histopathology image, and causing a user interface to display the output image.

Some embodiments provide for at least one non-transitory computer readable storage medium, storing processor executable instructions, that when executed by at least one computer hardware processor cause the processor to perform a method including: obtaining the histopathology image, the image containing histopathology data of one or more tissue samples, processing the histopathology image data using at least one trained machine learning model to determine a likelihood of each of a plurality of diagnostic entities being present in the one or more tissue samples, determining an output set of diagnostic entities by applying a conformal prediction technique to the determined likelihoods, wherein the size of the output set determined by the conformal prediction technique is determined based on the determined likelihoods, generating an output image by applying indications of a presence of the output set of diagnostic entities to the histopathology image, and causing a user interface to display the output image.

### BRIEF DESCRIPTION OF DRAWINGS

FIG. 1A is an example environment to which the technology described herein may be deployed, according to some embodiments of the technology described herein.

FIG. 1B is an example process which may be performed by a histopathology image analysis system, according to some embodiments of the technology described herein.

FIG. 2 illustrates a slide depicting a skin biopsy prior to and following application of automated orientation techniques, according to some embodiments of the technology described herein.

FIG. 3 illustrates the steps of a representative method for performing automatic orientation of a slide, according to some embodiments of the technology described herein.

FIG. 4 illustrates rotations applied to histopathology images by pathologists before and after automatic rotations are applied to the histopathology images.

FIG. 5 is an example of data which may be provided on a user interface of a user device during a review, according to some embodiments of the technology described herein.

FIG. 6 is an example of a field of interest which may be provided on a user interface during a review, according to some embodiments of the technology described herein.

FIG. 7 is an example of an overlay which may be provided on a user interface during a review, according to some embodiments of the technology described herein.

FIG. 8 is an example of a user interface of a histopathology analysis system including recommended stains.

FIG. 9 is an example of a user interface of a histopathology analysis system including a lesion depth measurement, according to some embodiments of the technology described herein.

FIG. 10 is an example of determining whether to perform a margin assessment on a histopathology image, according to some embodiments of the technology described herein.

FIG. 11 is an example of a workload display of a user interface of a histopathology image analysis system, according to some embodiments of the technology described herein.

FIG. 12 is an example process for preparing a histopathology image for analysis, according to some embodiments of the technology described herein.

FIG. 13 is an example process for analyzing a histopathology image, according to some embodiments of the technology described herein.

FIG. 14 is an example process for analyzing a histopathology image, according to some embodiments of the technology described herein.

FIG. 15 illustrates an example of a backbone architecture for use in an example machine learning model, according to some embodiments of the technology described herein.

FIG. 16 illustrates an example of a model including a backbone and multiple adaptation heads of an example machine learning model, according to some embodiments of the technology described herein.

FIG. 17 shows a block diagram of an exemplary computing device, according to some embodiments of the technology described herein.

### DETAILED DESCRIPTION

There is an increasing global demand for pathologists due to the ever-growing number of medical imaging and diagnostic procedures being performed. For example, the number of skin biopsies collected in the United States increases at an annual rate of 6%, building on a 154% increase from 1986 to 2001. Despite this rising need, the number of practicing pathologists has declined over recent years, creating a significant gap between supply and demand. Recognizing this challenge, the inventors have recognized and appreciated the critical importance of enhancing the productivity of individual pathologists. By enabling each pathologist to review more slides within a given day, the efficiency of diagnostic workflows can be improved, ultimately addressing the shortage and ensuring timely and accurate diagnostic services for patients.

Pathologists review histopathology specimens (e.g., digital WSIs) to identify the presence of one or more diagnostic entities within the specimens and to diagnose patients based on their reviews. Because of the ever increasing volume of specimens to review, it is important for pathologists to quickly and accurately review specimens. In any given day, a pathologist can waste several minutes when reviewing hundreds of images per day if they are delayed by several seconds when reviewing each image. This can negatively affect the pathologist's productivity.

Furthermore, it is important for pathologists to return diagnoses as fast as possible, so patients may receive any necessary treatment. In many healthcare settings (e.g., hospitals, doctor offices and specialty clinics) staining & imaging of tissue samples for analysis by pathologists occurs at set intervals (e.g., daily, every 6 hours, every 4 hours, among other time intervals). Therefore, if a pathologist desires to obtain additional stains or images of a particular specimen, they must request this work early in the day in order to receive the results before the end of the day. Thus, it is important for pathologists to prioritize review of complex specimens or specimens which are more likely to require additional workup (e.g., laboratory work such as staining and imaging).

Accordingly, the inventors have developed techniques for facilitating the review of histopathology images by pathologists. The techniques described herein optimize images for review by pathologists. In some embodiments, the techniques described herein provide pathologists with a histopathology image and an output set of predicted diagnostic entities present in the modified image. Pathologists may use the set of predicted diagnostic entities during review to assist in determining the diagnostic entities present in a specimen. Pathologists may additionally use the set of predicted diagnostic entities before their review to prioritize their reviewing schedule, such that cases more likely to require additional testing are reviewed earlier in the day. This may result in faster diagnoses as the additional testing can be obtained and reviewed on the same day, whereas without the indications of potentially complex cases, such prioritization would not be possible or practical.

In some embodiments, the techniques described herein may be implemented in a histopathology image analysis system. A histopathology image analysis system may be implemented in a healthcare environment and accessible through one or more connected devices. A histopathology image analysis system may include one or more modules to implement the techniques described herein. A histopathology image analysis system may provide output data for review by a pathologist. The output data may include indications of one or more diagnostic entities present in a histopathology image, and/or a histopathology image with applied indications of diagnostic entities, among other data. A histopathology image analysis system may provide a user interface for a user to review output data.

The histopathology images analyzed may be obtained by imaging sections of a tissue sample obtained from resection or biopsy. The samples may be stained for analysis, for example using an H&E or similar stain. The images may be obtained via a microscope, for example, images at 20× and/or 40× magnification. Images may be obtained using whole slide imaging scanners such as Aperio AT2, Aperio GT450, Hamamatsu S360, Philips UFS, or Ventana DP200, among other scanners. The images may be digital images stored in data structures, for example .bif(f), .isyntax, .mrxs, .ndpi, .svs, and/or .tif(f) files.

According to some embodiments, the inventors have developed techniques for automatic rotation of histopathology images in accordance with the orientations preferred by pathologists. Precise orientation of tissue specimens within slides prior to imaging is difficult due to the size and fragility of the samples. On average, between 60% and 90% of all slides require rotation to ensure that the image is properly orientated. Therefore, the inventors have developed techniques for automatically rotating histopathology images, which reduces the time and input required by a pathologist while reviewing a sample. In some embodiments, the techniques involve isolating a target tissue from a histopathology image, identifying a contour of the target tissue, identifying an axis associated with the contour and determining a rotation to be applied to the histopathology image based on the axis. Using the techniques developed by the inventors and described herein, the number of slides with orientations between −20 degrees and 20 degrees is increased from about 10% of the total number of slides (corresponding to a random distribution of orientations) to approximately 70%. This represents a significant improvement in that it decreases the time that pathologists need to spend rotating slides, thus reducing the number of required computer input interactions and therefore increasing their productivity and reducing their required time to process slide information. The techniques described herein are expected to save 40-80 hours of work annually per pathologist, when extended across a typical workload.

In some embodiments, when automatically rotating a histopathology image, isolating the image data corresponding to the target tissue may involve: determining tissue types of the one or more tissue samples depicted in the histopathology image, and isolating the target tissue based on the tissue types of the one or more tissue samples. For example, a histopathology image may include multiple tissue samples which include multiple tissues. The target tissue may be predetermined (e.g., by a user such as a pathologist) and isolated to determine the rotation to be applied, such that the histopathology image is oriented to facilitate review of the target tissue. In some embodiments one or more ML models are used to identify the tissue type(s) within a histopathology image.

In some embodiments, isolating the target tissue involves masking the histopathology image to remove the background and/or small objects from the image. For example, Otsu masking, triangle masking, minimum error thresholding, Li's thresholding method, Yen's thresholding method, or other automatic thresholding methods may be used to mask the target tissue from the histopathology image. After, masking a connected components analysis may be performed on the masked histopathology image to identify components (e.g., the individual tissue samples) within the histopathology image. These components may be isolated as the target tissues within the histopathology image. In some embodiments one target tissue is isolated from the histopathology image. In some embodiments multiple target tissues are isolated from the histopathology image.

In some embodiments a contour of isolated target tissues is determined. The contour of the target tissues may correspond to the border of the isolated target tissues having a certain width, such as through edge detection of the isolated target tissue. For example, the contour of the target tissue may be the image data corresponding to the border of the isolated target tissues having a width of 1 pixel, 2 pixels, 3 pixels, between 1-5 pixels, between 1-10 pixels, between 1-20 pixels, or any other suitable width.

In some embodiments, identifying the axis associated with the contour of the target tissue involves determining a shape of the contour and determining the axis based on the shape. For example, the shape of the contour may be a shape bounding the contour. The shape of the contour may be the ellipse with the smallest area that bounds the contour. In such examples, the axis may be the major or minor axis of the ellipse.

In some embodiments, the predetermined preferred orientation for a histopathology image is determined based on the type of the target tissue. For example, the predetermined preferred orientation may be determined based on the consensus pathologist preference for a particular tissue type.

As described above, the preferred orientation for a tissue sample obtained during a skin biopsy has the epidermis positioned at the top of the histopathology image, therefore this preferred orientation may be determined based on an identified epidermis in an image. In such embodiments, the location of the epidermis within the target tissue (or each of the target tissues when multiple target tissues) may be determined. The location of the epidermis may be determined by the pixel colors of the medical imaging data of the isolated target tissues. For examples, the tissues within the histopathology image may have a H&E stain applied, and the epidermis will correspond to the darkest portions of the histopathology image, which in H&E stained tissues are purple. Therefore, the location of the epidermis may be determined based on the pixel colors of the image data corresponding to the isolated target tissues, for example the RGB or HEX values of the pixels may be determined, and the most purple or darkest pixels are identified as the epidermis. To identify the position of the minor axis to the location of the epidermis in a tissue sample, all points of the contour may be projected onto the identified axis, and the section of contiguous points in the contour, which is half of the contour length, that maximizes the dark or purple pixels is identified as the “top” portion of the contour. The rotation to be applied to the histopathology image may be determined as the rotation that orients the minor axis of the ellipse bounding the contour vertically and has the half of the contour containing the epidermis oriented at the top of the histopathology image.

In some embodiments, when multiple target tissues are isolated, the rotation to be applied to the histopathology image may be determined as a weighted average of the rotations determined for each of the target tissues. For example, the rotation to be applied may be determined by weighting the rotations based on the areas of the target tissues, such that the largest tissue portions contribute most to the rotation to be applied to the histopathology image.

As described above, an output set of predicted diagnostic entities may be displayed to a user in addition to a histopathology image. In some embodiments, the output set of predicted diagnostic entities is determined in part using a trained machine learning model. In some embodiments, the trained machine learning model may be configured to output a probability of a particular histopathology image containing a particular diagnostic entity, for each of the entities the model is trained to identify. The inputs to the ML model may include a histopathology image and/or a rotated histopathology image. In some embodiments, the trained machine learning model is a neural network trained on labeled histopathology images. In some embodiments, the trained machine learning model is a neural network trained on labeled histopathology images configured to output probabilities using a softmax function. In some embodiments, the trained machine learning model is an additive multiple instance model. In some embodiments, other machine learning models capable of image classification may be used, such as transformer-based models fine-tuned for identifying diagnostic entities in histopathology images, among other types of ML models.

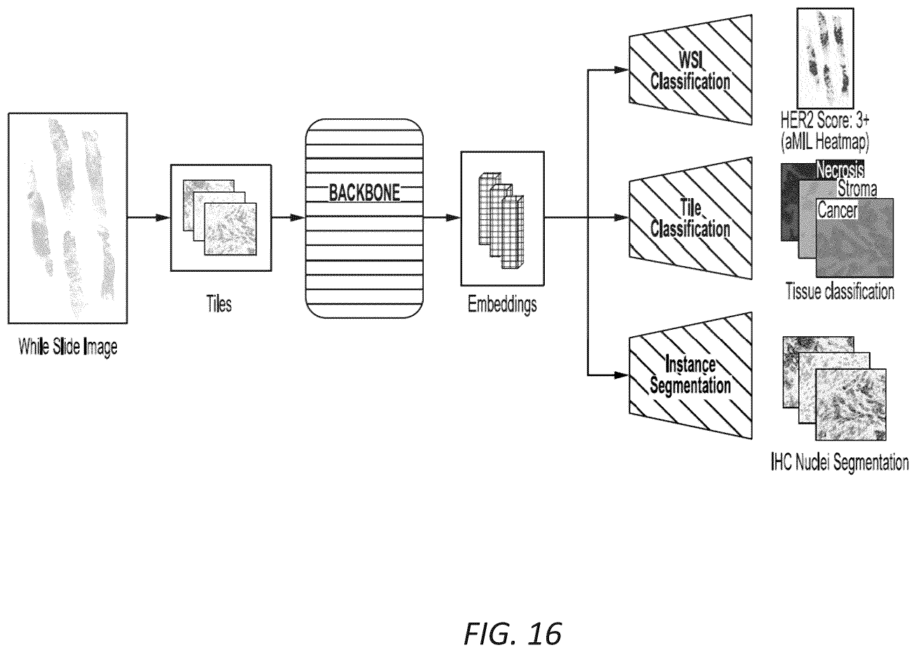

In some embodiments, the trained ML model is a foundation model configured to identify features indicative of diagnostic entities within histopathology images. Foundation models are large scale deep learning models that are pre-trained on broad-scale, unlabeled data using self-supervision. The foundation model may include a backbone and one or more adaptation heads adapted to specific tasks. An example model is shown in FIG. 16. Examples of ML models which may be used to analyze histopathology images, are described in U.S. Patent Pub. No.: US2025/0336065 entitled MULTI-RESOLUTION FOUNDATION MODEL FOR PATHOLOGY, which is incorporated by reference herein in its entirety. Additional examples of ML models are described in Juyal et al., PLUTO: Pathology-Universal Transformer, arXiv:2511.02826, and in Javed et al., Additive MIL: Intrinsically Interpretable Multiple Instance Learning for Pathology, arXiv:2206.01794, both of which are incorporated by reference herein in their entirety.

The ML model may include a backbone that is pre-trained on a diverse dataset from multiple sites and that extracts meaningful representations across different levels of the Whole Slide Image (WSI) pyramid (e.g., slide, tissue and cellular level features). The backbone may be the primary component of the ML model responsible for extracting features from the input data (e.g., histopathology images). This part of the model involves several layers of a neural network (or more than one neural network) that process the input data to create a representation or set of features (e.g., embeddings) that encapsulate the important information needed for further tasks. The layers may be layers of a transformer in some embodiments. Alternatively, the layers may be layers of a convolutional neural network (CNN) that process images to detect edges, textures, and other visual elements. However, other types of neural networks are possible.

In some embodiments, the backbone is based on the DINOv2 model, which combines DINO and iBot losses and a KoLeo regularizer to learn relevant representations at the tile and patch levels. A masked autoencoder (MAR) may be used to capture details of objects at different levels of granularity. The MAE may reconstruct masked regions of the input image (often a large fraction of the input) from the unmasked regions. Masking may be performed by varying the patch sizes used for masking while using images across different resolutions of the WSI. In addition to the pixel-level reconstruction loss used in MAE, a Fourier reconstruction loss may be used to control the amount of low- and high-frequency information preserved during the pre-training process.

The FlexiViT technique may be used to enable the encoder and decoder of the backbone to handle varying patch sizes for multi-scale masking. Since patch size controls the granularity of information captured by the encoder, different downstream tasks may need different patch sizes for optimal performance. The FlexiViT setup allows the backbone to be adapted to different tasks without needing to train individual backbones for every patch sizes. The patch size may also determine the effective sequence length used in ViTs and FlexiViT which allows for the memory usage of the model to be catered to select the most suitable patch size at inference time.

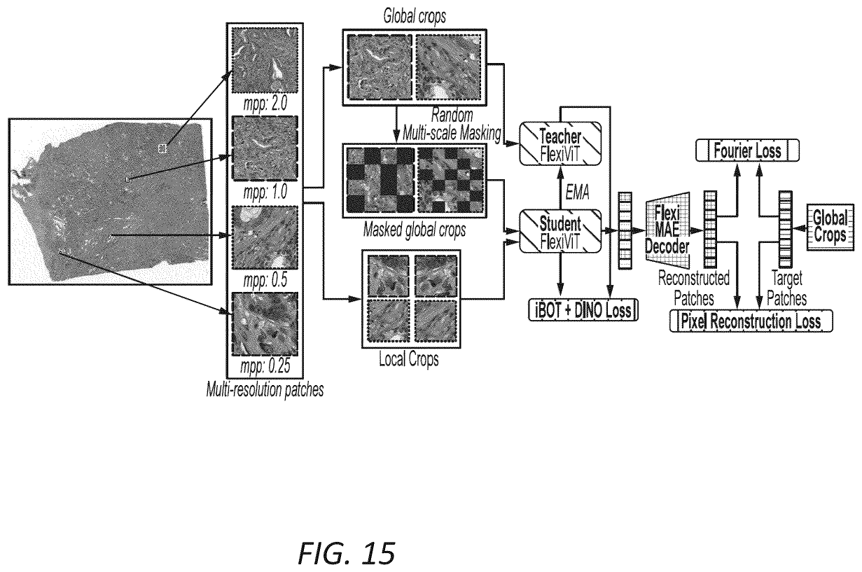

The backbone may be pretrained using a set of WSIs. The image resolution may be selected randomly (e.g., with prespecified probabilities). Global crops (e.g., two) and local crops (e.g., four) of suitable sizes (e.g., 224 and 96 respectively) may be taken from each image, consistent with DINOv2 training, which may be passed to students and teachers for pretraining. The crops provided to the student may be randomly masked for the iBOT objective. Further, a separate masking setup with a higher masking ratio may be applied to the global crops for the MAE objective. Beyond the original MAE objective, the reconstructed image may be decomposed into its low- and high-frequency components. For example, the Fourier spectrum of the reconstructed image may be dissected into low-frequency and high-frequency bands using a set of low-pass and high-pass filters. After this separation, the L2 loss may be computed independently for both the low- and high-frequency parts of the image. The sum of these weighted losses forms the overall Fourier reconstruction loss, which the training process aims to minimize. An example backbone is shown in FIG. 15.

In some embodiments, task-specific adaptation heads may be added to the backbone and adapted through supervised fine-tuning, while keeping the backbone fixed (frozen). The adaptation heads may be adapted to various tasks, for example slide level, tissue level, cellular level, and subcellular level tasks. Slide-level task adaptation may involve performing weak supervision on slide-level labels of histopathology images, for example by using a MIL model. Adaptation to tissue-level and cellular/subcellular-level biological scales may be obtained through fine-tuning either a tile classification or an instance segmentation adaptation head.

In some embodiments the trained machine learning model may be trained to identify one or more entities present in a histopathology image. For example, the trained machine learning model may be trained to identify one or more of: benign nevus, basal cell carcinoma, squamous cell carcinoma, seborrheic keratosis, actinic keratosis, dermatitis, dysplastic nevus, squamous cell carcinoma in situ, verruca vulgaris, benign non-melanocytic lesion, scar, cyst, lichenoid keratosis, melanoma in situ, vascular lesion, invasive melanoma, and/or normal skin.

The outputs of the ML model may include: indications of one or more diagnostic entities present in an input histopathology image, and/or locations of image features corresponding to and/or indicative of particular diagnostic entities or features within the histopathology image. Heatmaps indicating the location of features corresponding to and/or indicative of particular diagnostic entities or features within the histopathology image may be output by the ML model and/or generated from the outputs of the ML model.

Conventional techniques for predicting disease states or other features from histopathology images output probabilities associated with each of multiple diseases or features the techniques are trained to identify. Such techniques provide pathologists with a list of possible diagnoses for a particular histopathology image, many of which do not reflect the actual condition of the associated patient. These lists, with inaccurate diagnoses, can slow review by pathologists, for example by turning their focuses to potential diagnoses which are not present in the histopathology image. Accordingly, the inventors have developed techniques for controlling the diagnostic entities output to users, for example by using calibrated and/or conformal prediction techniques. For example, some embodiments provide for a method for preparing a histopathology image for analysis by processing the histopathology image data using at least one trained ML model to determine the likelihoods of one or more diagnostic entities being present in the one or more tissue samples depicted in the histopathology image, determining a calibrated probability for each of the determined likelihood, and generating an output set of diagnostic entities, the size and entities within the set are determined based on the calibrated probabilities. In some embodiments, the output set of diagnostic entities is displayed to users (e.g., pathologists) on a user interface.

In some embodiments, the probabilities of diagnostic entities output by the one or more ML models may be confidence calibrated using one or more calibration techniques to determine calibrated probabilities of diagnostic entities being present in a histology image. For example, post-hoc calibration techniques may be used to adjust the probabilities output by a ML model (e.g., from a softmax layer of the ML model), after training of the model, to ensure the probabilities align with the actual performance of the model. Calibration of a ML model may involve holding out labeled data from the training dataset for use in calibration, and determining calibration parameters by processing the data using the ML model. The calibration parameters are then applied to outputs of the ML model. Examples of calibration techniques that may be used include temperature scaling, vector scaling, or Dirichlet calibration. Using these techniques, the probabilities output by the ML model are calibrated according to the confidence of the model to generate calibrated probabilities.

In some embodiments, the output set of entities to display to a user are determined from the probabilities output from the machine learning model. The output set of entities may be determined using a conformal prediction technique. In some embodiments, where outputs of the ML model are calibrated, the calibrated confidences may be used to determine the output set of entities. The conformal prediction technique may be tailored to provide a desired coverage for the determined set of entities. The coverage is the probability that a particular output set of entities contains a correct diagnostic entity for a specimen. In some embodiments, coverage is set by a user and may be set at any desired value for example, 0.75, 0.8, 0.85, 0.9 0.95, 0.99 or any other value. In some embodiments, the size of the output set of diagnostic entities is determined using a conformal prediction technique according to a desired coverage. For example, the smallest set of entities achieving the desired coverage is output.

In conformal prediction techniques, desired coverage is related to the set size that can be output, with larger set sizes typically having higher coverage. A smaller set size is desirable for review by a pathologist, as smaller sets of entities are easier to review and can focus the pathologist's review on fewer diagnostic entities. However, smaller set sizes will typically have lower coverage. Therefore, it is important to balance the set size with the coverage when using conformal prediction techniques. In some embodiments, the output set size is determined by a user. For example, the output set size may be limited to one diagnostic entity, two diagnostic entities, three diagnostic entities, four diagnostic entities, five diagnostic entities, up to 10 diagnostic entities or up to 15 diagnostic entities. In some embodiments, a maximum set size is set by a user and the conformal prediction technique outputs the smallest set with the desired coverage, or, if the smallest set achieving the desired coverage is greater than the maximum set size, the output set is the set with the maximum set size and the greatest coverage according to the conformal prediction technique.

In some embodiments, different conformal prediction techniques may be used to determine the output set. In some embodiments, softmax function outputs from a machine learning model may be used to determine the output set, with scores determined from the probabilities, a quantile determined from the scores and the output set determined based on the quantile. For example, conformal prediction may be used with a ML model trained to output probabilities of particular diagnostic entities in a histopathology image. The ML model may be calibrated or uncalibrated. A set of labeled data, not used during training of the ML model may be used to calibrate the model for conformal prediction. The ML model may output a set of probabilities for each of the histopathology image in the set of labeled data, and a score function may be used to determine the scores from each of the probabilities. The score function may determine the score for each respective probability as one minus the softmax output of the true class. The quantile of the calibration scores is then determined, and this quantile is used for future predictions by the ML model, where each probability is scored using the score function and is included in the output set if the score exceeds a threshold determined using the quantile.

In some embodiments, the output set is determined using adaptive prediction sets, where probabilities output by a machine learning model are sorted and cumulative probabilities of sets containing a correct output are determined. A quantile is determined based on the cumulative probabilities, and the output set is determined based on the quantile. For example, conformal prediction may be used with a ML model trained to output probabilities of particular diagnostic entities in a histopathology image. The ML model may be calibrated or uncalibrated. A set of labeled data, not used during training of the ML model may be used to calibrate the model for conformal prediction with adaptive prediction sets. A score function may be used to determine the scores from each of the probabilities output by the ML model from the set. The score function may sort the probabilities from most to least likely and greedily sum the probabilities until the set contains the true label. The quantile of the calibration scores is then determined, and this quantile is used for future predictions by the ML model, where for each of the scores, the score is included in the output set if the score exceeds a threshold determined using the quantile.

In some embodiments, a regularized adaptive prediction set technique is used, where a set size regularization term is introduced to control the size of the output set. This is beneficial in some cases because adaptive prediction sets may be sensitive to noisy probability estimates of classes with lower probabilities. In a regularized adaptive prediction set technique, the score function may introduce a regularization term to limit the sizes of output sets generated using the technique. For example, the regularization term may apply a penalty to each score for a particular probability associated with a diagnostic entity, which is below a threshold score.

In some embodiments conformal prediction techniques may be used to generate atypicality-aware prediction sets. Such atypicality-aware prediction sets may be generated when using an adaptive prediction set technique and/or a regularized adaptive prediction set technique. For example, when calibrating for prediction, using unseen, labeled data, the probabilities output by the ML model may be grouped according to their confidence and atypicality quantiles. A group may be defined by four thresholds: the lower and upper atypicality bounds, and the lower and upper confidence bounds. Then, separate thresholds are fit for each group's prediction sets by using adaptive prediction set technique and/or a regularized adaptive prediction set technique as a sub routine. This allows for lows for an adaptive threshold depending only on the atypicality and confidence of predictions.

In some embodiments, class conditional coverages may be used with conformal prediction techniques. The inventors have appreciated that when using marginal coverage, conformal prediction methods may substantially undercover some diagnostic entities and may overcover other diagnostic entities to achieve the desired coverage. Classwise conformal prediction ensures that for each diagnostic entity the probability of being included in the output set when it is the true label is greater than a threshold. Classwise conformal predictions may be accomplished by stratifying scores determined from probabilities output by the trained machine learning model by diagnostic entity, within each diagnostic entity determining the conformal quantile, and iterating through the diagnostic entities and determining whether to include them in the output set based on the associated quantile. In some embodiments, clustered conformal prediction techniques may be used to determine the output set to improve the likelihood of the correct diagnostic entity being included in the output set. In some embodiments, clustered conformal prediction is accomplished by clustering entities based on the similarity of the conformal score distributions. K-means clustering or other clustering methods may be used to group entities, and conformal prediction is applied at the cluster level to determine the output set of entities. For example, for each cluster a single cluster-level quantile is determined based on all of the data in the cluster. When constructing prediction sets, a class is included in the set of the conformal score for the class is less than the quantile that contains the class. When using a clustered conformal prediction technique, a null cluster may be used to handle classes which are rare or lack sufficient data. When constructing prediction sets, for classes assigned to the null cluster, the conformal score is compared against the quantile that would be obtained from running standard conformal prediction on the proper calibration set (e.g., without clustering).

The techniques developed by the inventors, which involve combining a trained machine learning model with conformal predictions to determine an output set of diagnostic entities for a particular histopathology images have been tested to determine their performance. A panel of 11 board-certified pathologists were asked to review 519 histopathology images and to determine diagnostic entities present in these images. Each slide was reviewed and labeled by exactly 5 pathologists. The pathologists only defined a clear majority diagnosis in 80% of the images (3 or more pathologists agreeing on a single diagnosis). In a similar test, the conformal prediction techniques developed by the inventors were able to generate output sets including the majority diagnosis entity in 88% of cases where there was a majority diagnosis, demonstrating the techniques developed by the inventors are accurate and can assist pathologists during review to determine a correct diagnosis. Furthermore, the techniques developed by the inventors have been shown to accurately identify about 90% of possible diagnostic entities in a standard lab's volumes; distinguish between neoplastic, inflammatory, infectious and normal lesions; distinguish between types of neoplastic lesions (e.g. basaloid, squamous, and melanocytic neoplastic) and distinguish between types of melanocytic lesions (e.g. benign nevus, dysplastic nevus, MIS and melanoma). Most cases analyzed by the model (77%) had a conformal prediction set length of 3, while 16% had a conformal prediction set length of 1, indicating extremely high model confidence for the cases in the latter group. Furthermore, the accuracy of the single top model prediction was 74.25%, which increased to 85.75% and to 89.75% when assessing the accuracy of the top 2 and 3 model predictions, respectively.

The conformal prediction techniques were additionally tested and found to correlate with consensus pathologist analysis. A foundational ML model with a transformer-based backbone and adaptation heads adapted for histopathology image analysis analyzed the above-described dataset and the output sets were determined using adaptive prediction sets. The model was trained on a set of whole slide images to identify 17 dermatopathology entities. The conformal prediction set size was tested for sizes of 1, 2 and 3 predictions. The model accurately distinguished between the broad categories of neoplastic, inflammatory, and normal classifications, with an overall percent agreement of 91% for the top predicted class. Compared to the coverage of the consensus label in the prediction set (88.4%), the accuracy of the single top model prediction was 66.1%. The majority of cases examined (62.8%) had more than one unique label across the 5 pathologists; in other words, there was disagreement among pathologists for nearly ⅔ of cases. The number of pathologist majority votes correlated with the model confidence had Pearson r=0.493, p<0.001. For cases with a 4-1 or 3-2 majority, the minority label was also often present in the prediction set. Therefore, the model showed strong accuracy and the output sets had high coverage of consensus pathologist labels despite the large number of possible classifications and disagreement among pathologists.

Overall, the performance for lesion classification of the models described herein, leveraging ML techniques and conformal predictions, was determined to be comparable to that of human experts (Table 1 below). When considering diagnoses representing ~95% of typical dermatopathology case volumes (N=251), compared to the five-pathologist reference consensus, the model achieved a Cohen's kappa value of 0.630, which is on par with the observed inter-pathologist agreement in this dataset (Cohen's kappa=0.640). Similar accuracy metrics were observed for the model and pathologists, as well. OPA, PPA, and NPA scores for pathologists were 0.672, 0.578, and 0.980, respectively, compared to model OPA, PPA, and NPA values were 0.663, 0.566, and 0.980, respectively. Thus, model predictions are comparable to pathologist assessment when considering the most commonly seen types of cases, indicating it can perform at the level of a trained pathologist. Notably, in broad categorization tasks (e.g., distinguishing neoplastic vs. inflammatory vs. normal tissue), the model achieved similar overall percent accuracy to pathologists (77.9% compared to 76.7%), These results suggest that the models described herein can reliably recognize when a biopsy is benign/inflammatory versus malignant—a crucial safety aspect for a histopathology image triage tool. Furthermore, the models demonstrated comparable accuracy to pathologists for distinguishing between basaloid, squamous, and melanocytic neoplastic lesions, as well as for distinguish between subtypes of melanocytic lesions (benign nevus, dysplastic nevus, melanoma in situ [MIS], and melanoma).

TABLE 1
Comparison of model performance to human expert pathologist

| | | | Point | Non- |
|---|---|---|---|---|
| | Pathologist- | Model- | Difference | Inferiority |
| | Pathologist | Pathologist | (Model- | Criteria* |
| Objective | (95% CI) | (95% CI) | Pathologist) | Met? |
| Accurate- triage | κ: 0.640 | κ: 0.630 | −0.010 | Yes |
| of ~95% of | (0.629, | (0.606, | | |
| skin lesions | 0.653) | 0.653) | | |
| (by case volumes) | OPA: 0.672 | OPA: 0.663 | −0.009 | Yes |
| (N = 251 WSIs) | (0.661, | (0.641, | | |
| | 0.683) | 0.685) | | |
| | PPA: 0.578 | PPA: 0.566 | −0.012 | Yes |
| | (0.562, | (0.537, | | |
| | 0.594) | 0.593) | | |
| | NPA: 0.980 | NPA: 0.980 | 0.000 | Yes |
| | (0.979, | (0.979, | | |
| | 0.981) | 0.981) | | |
| Distinguishment | κ: 0.440 | κ: 0.398 | −0.042 | Yes |
| between | (0.426, | (0.368, | | |
| neoplastic, | 0.453) | 0.427) | | |
| inflammatory, | OPA: 0.767 | OPA: 0.779 | 0.012 | Yes |
| infectious, and | (0.760, | (0.765, | | |
| normal lesions | 0.774) | 0.792) | | |
| (N = 519 WSIs) | PPA: 0.540 | PPA: 0.494 | −0.046 | Yes |
| | (0.529, | (0.479, | | |
| | 0.552) | 0.510) | | |
| | NPA: 0.868 | NPA: 0.842 | −0.026 | Yes |
| | (0.864, | (0.835, | | |
| | 0.872) | 0.850) | | |
| Distinguishment | κ: 0.755 | κ: 0.689 | −0.066 | Yes |
| between basaloid, | (0.739, | (0.652, | | |
| squamous, and | 0.772) | 0.727) | | |
| melanocytic | OPA: 0.859 | OPA: 0.830 | −0.029 | Yes |
| neoplastic lesions. | (0.850, | (0.809, | | |
| (N = 180 WSIs) | 0.868) | 0.852) | | |
| | PPA: 0.851 | PPA: 0.777 | −0.074 | Yes |
| | (0.840, | (0.755, | | |
| | 0.861) | 0.799) | | |
| | NPA: 0.916 | NPA: 0.884 | −0.032 | Yes |
| | (0.911, | (0.870, | | |
| | 0.922) | 0.897) | | |
| Distinguishment | κ: 0.623 | κ: 0.607 | −0.016 | Yes |
| between | (0.607, | (0.575, | | |
| melanocytic | 0.638) | 0.642) | | |
| lesions: | OPA: 0.698 | OPA: 0.686 | −0.012 | Yes |
| benign nevus, | (0.685, | (0.658, | | |
| dysplastic nevus, | 0.711) | 0.714) | | |
| MIS, melanoma. | PPA: 0.600 | PPA: 0.568 | −0.032 | Yes |
| (N = 160 WSIs) | (0.587, | (0.544, | | |
| | 0.613) | 0.593) | | |
| | NPA: 0.939 | NPA: 0.937 | −0.002 | Yes |
| | (0.937, | (0.931, | | |
| | 0.942) | 0.942) | | |

The techniques developed by the inventors, including the automated orientation of histopathology images and the use of conformal and calibrated techniques for predicting diagnostic entities in a histopathology image, may be used to prepare data for display on a user interface. The user interface may be included as part of a histopathology image analysis system (e.g., on one or more devices included within the system). The data displayed may include one or more of: a histopathology image, one or more modifications to the histopathology image, one or more predicted diagnostic entities present in the histopathology image (e.g., an output set generated using conformal prediction), one or more recommended actions for further processing of the histopathology image, among other data described herein. The data displayed on the user interface, generated using the techniques described herein, simplifies the review process for pathologists and decreases the time needed to review a histopathology image. In some embodiments, a modified histopathology image is displayed to a user in order to indicate the presence of one or more diagnostic entities in an output set of diagnostic entities to the user. In some embodiments the modified histopathology image is a histopathology image with one or more features indicative of diagnostic entities rendered over the image. In some embodiments, the histopathology image itself may not be modified by the histopathology image analysis system but may appear to a user of the system to be modified based on the one or more features rendered over the image. In some embodiments, a user may be able to toggle on and off the features indicative of the one or more diagnostic entities.

In some embodiments, diagnostic entities may be indicated through one or more fields of interest (FOIs) applied to the histopathology image. The fields of interest may be determined based on the likelihood of a particular diagnostic entity being present within that portion of the histopathology image. In some embodiments, the fields of interest may be determined based on the outputs of the trained machine learning model. In some embodiments, the fields of interest are determined based on outputs of a trained machine learning model separate from that used in determining the likelihoods of the diagnostic entities being present in the histopathology image. In some embodiments the fields of interest are determined based on the output set of diagnostic entities. The fields of interest may be areas of the histopathology image which are enlarged or surrounded by a border such that a reviewer may focus on these areas while reviewing the histopathology image.

In some embodiments, the one or more ML models may output feature maps corresponding to locations of features corresponding to and/or indicative of one or more diagnostic entities within the histopathology image and/or areas of background. The FOIs may be determined using such feature maps. For example, to determine the FOIs, a candidate grid with may be generated over regions of tissue in the output feature map. The grid may have specific dimensions based on the size of tissue being analyzed, for example having a length and/or width between 100-10,000 μm, with a spacing between 10-1000 μm between each gridline. An area threshold operation may then be performed to identify portions of the histopathology image indicative of particular diagnoses. For example, if a portion of the grid has greater than a threshold area having a substance of interest (e.g., a particular feature and/or substance indicative of one or more diagnostic entities), the area may be designated as a potential FOI. Conversely, if portion has less than a threshold area of a substance of interest, it may be removed from consideration as a FOI. The threshold may be any suitable area, for example between 500 μm²-5000 μm². Each of the potential FOIs may be sorted based on the area of the substance of interest within the potential FOIs, and a predetermined number of the top FOIs may be selected for each of the diagnostic entities (e.g., those entities within the output set of entities), for example the top 1, 2, 3, 4, 5, or greater than 5 potential FOIs may be identified for each entity. A limit on the total number of FOIs for the histopathology image may be implemented, for example up to 5, up to 10, up to 15, up to 20 or greater than 20 FOIs. In some embodiments, a spacing constraint may be implemented such that there is greater than a threshold distance between the centers of potential FOIs, for example greater than 1 mm, greater than 2 mm, greater than 3 mm, greater than 4 mm, or greater than 5 mm. In some embodiments, potential FOIs may be merged into a single FOI, for example if two or more potential FOIs overlap by greater than a threshold amount (e.g., greater than 10%, greater than 20%, greater than 30%, greater than 40%, greater than 50%, or any suitable level of overlap).

The techniques described herein have been used to accurately identify portions associated with diagnostic entities within histopathology regions. As shown in table 2 below, the fields of interest identified in histopathology images are identified at rates aligned with the confidence levels determined for the entity being present in the fields of interest. This indicates the fields of interest are accurate and can assist pathologists in identifying diagnostic entities present in histopathology images.

TABLE 2

| Entity | | | | | Accep- |
|---|---|---|---|---|---|
| Confi- | # of Fields of Interest | | | | tance |
| dence | Evaluated | Accepted | Uncertain | Rejected | Rate |
| [80, 100%) | 70 | 64 | 2 | 4 | 94% |
| [60, 80%) | 60 | 40 | 2 | 28 | 52% |
| [40%, 60%) | 50 | 19 | 11 | 20 | 49% |
| [20%, 40%) | 40 | 11 | 0 | 29 | 28% |
| [0%, 20%) | 30 | 6 | 1 | 23 | 21% |

In some embodiments, overlays are applied to the histopathology image to indicate the presence of a diagnostic entity within the image. In some embodiments, the opacity of the overlay may vary to indicate the likelihood of portions of the overlay corresponding to a particular diagnostic entity. For example, the opacity of an overlay may be increased in regions where it is more likely that a diagnostic entity is present, such as based on the presence of one or more features and/or substances of interest within the histopathology image corresponding to and/or indicative of the diagnostic entity. In some embodiments, multiple overlays may be applied to an image. In some embodiments, multiple overlays may be applied to an image corresponding to different occurrences of the same diagnostic entity within the histopathology image. In some embodiments, multiple overlays may be applied to an image corresponding to regions of the image associated with different diagnostic entities. In some embodiments, multiple overlays may have different colors to assist users in differentiating between diagnostic entities and/or occurrences of a diagnostic entity.

In some embodiments, data related to a histopathology image may be provided on a histopathology image. In some embodiments, the data may be generated by the histopathology image analysis system and provided responsive to a user request. In some embodiments, a histopathology image analysis system may provide measures typically generated by a pathologist as part of synoptic reporting for a diagnosis. For example, in some embodiments, a histopathology image analysis system may provide measurements of mitotic rate. In some embodiments, mitotic rate data may be determined using one or more machine learning models trained to identify dividing cells in a tissue sample. In some embodiments, such data may include lesion depth. In some embodiments, lesion depth may be determined using known dimensions of a histopathology image. The inventors have appreciated that such data may assist in diagnosing diagnostic entities and may shorten the analysis and reporting time spent by a pathologist.

The techniques developed by the inventors have additionally increased the percentage of malignant diagnostic entity cases reviewable in the first half of a day. These cases include melanoma cases, basal cell carcinoma, and squamous cell carcinoma. In some embodiments, the techniques described herein are implemented via a histopathology image analysis system. Such systems may analyze histopathology images and provide one or more user interfaces for reviewing the images. Within a medical environment (e.g., hospital, hospital system or network, private practice, doctor's office, or other location), a pathologist may have a workload of cases to review. In some embodiments, the output set of diagnostic entities may be displayed to users on a user interface. In some embodiments, the output set of diagnostic entities are shown alongside a histopathology image. In some embodiments, the pathologist workload may be prioritized based on diagnostic entities associated with histopathology images (e.g., determined using the one or more ML models). For example, histopathology images associated with more severe diagnostic entities (e.g., malignant diagnostic entities) or diagnostic entities which require multiple rounds of testing (e.g., staining and imaging) may be prioritized for review (e.g., by positioning the entities at the top of a queue of entities to review or otherwise marking the cases as urgent). In some embodiments, the output set of diagnostic entities for a particular histopathology image are shown in a list view of available histopathology images for review. The techniques have been tested by simulating the workload for a pathologist and determining the difference in case rank for a default case order and a case order prioritized based on output sets determined for histopathology images. Using the techniques described herein, the percentage of true melanoma cases, basal cell carcinoma cases, and squamous cell carcinoma cases reviewable in the first half of the day increased from 51% to 69-73%. This improves the time to diagnosis for these cases and the likelihood that any required subsequent testing will be completed within the day.

The inventors have appreciated that techniques described herein may assist in the prioritization of pathology workloads and may assist in determining a diagnosis for a particular histopathology image. The inventors have appreciated that the output set alone may improve diagnosis accuracy by indicating the most likely diagnostic entities present in a specimen, diagnosis may additionally be improved through visual indications of diagnostic entities within the output set.

As discussed above, the inventors have appreciated that some specimens require additional analysis. For example, additional slices may be obtained from a specimen for imaging and analysis, and in some cases, additional slices of specimens may be stained using different stains from the original specimen that prompted the additional review. For example, H&E stain, Periodic Acid-Schiff (PAS) Stain, Masson's Trichrome Stain, Silver Stains, Immunohistochemistry (IHC) Stains, Cytokeratin stains, p40 stains, among other stains. Stains may be selected to highlight or show certain diagnostic entities or characteristics of a specimen.

In some embodiments, a histopathology image analysis system may determine one or more stains to recommend to a user for additional testing. In some embodiments, the one or more stains are determined based on probabilities output by the trained machine learning model, the output set of diagnostic entity, by a computer model and/or by a machine learning model separate from the trained machine learning model that outputs probabilities of diagnostic entities, among other techniques. In some embodiments, the one or more recommended stains may correspond to specific diagnostic entities and may be recommended to a user to confirm a diagnosis, for example, p40 and/or CK5/6 stains may be recommended to confirm diagnoses of some cancers. In some embodiments, a melan-A stain, a PRAME stain, and/or a SOX-10 stain may be recommended when dysplastic nevus is detected in a histopathology image. In some embodiments, a HMB45 stain, a Malan-A stain, a PRAME stain, and/or a SOX-10 stain may be recommended when an invasive melanoma is detected in a histopathology image. In some embodiments, a HMB45 stain, a Melan-A stain, a PRAME stain, and/or a SOX-10 stain may be recommended when melanoma in situ is detected in a histopathology image. In some embodiments, a CK5/6 stain and/or a p40 stain may be recommended when squamous cell carcinoma is detected in a histopathology image. In some embodiments, recommended stains are presented to users on a user interface. In some embodiments, recommended stains are presented alongside a histopathology image. In some embodiments, recommended stains are presented in a list view of available histopathology images.

In some embodiments, a histopathology image analysis system may determine a tumor margin assessment based on an analysis of a histopathology image. In some embodiments, a tumor margin assessment may be performed when cancer and margin ink are both identified in a histopathology image. In some embodiments, cancer may include detected diagnostic entities in a histopathology image, for example, basal cell carcinoma, squamous cell carcinoma, squamous cell carcinoma in situ, melanoma in situ, and/or invasive melanoma, among other types of cancers. In some embodiments, cancer may be determined based on probabilities of diagnostic entities output by a trained machine learning model, and/or the output set of diagnostic entities. In some embodiments, cancer may be determined using a machine learning model different from that configured to output probabilities of diagnostic entities. In some embodiments, margin ink may be determined based on the output of a machine learning model, the machine learning model configured to determine the probabilities of diagnostic entities, and/or a different model. The inventors have appreciated that by performing margin assessments on histopathology samples with both detected cancer and margin ink, the reviewing time of pathologists may be reduced.

In some embodiments, the trained ML model may output a feature map of features within the histopathology image including margin ink, usable tissue (e.g., as any tissue area that is well defined, where cells, structures and nuclei are easily identifiable and are free of imaging artifacts (e.g., scanning, coverslipping, sectioning, grossing/marker among other artifacts)) and cancer features. These outputs may be used to generate respective heatmaps representing the locations of cancer, margin ink and usable tissue within the histopathology image. An opening operation may be applied to the heatmap of margin ink, for example with a rectangular 3×3 kernel to remove small objects, then a dilation operation may be applied with a 5×5 kernel multiple times, for example between 3-5 times. A connected components analysis may be performed on the usable tissue heatmap to identify tissue sections of the histopathology image. For all connected components in the usable tissue heatmap, a contour (e.g., list of points at the edge of the usable tissue) is extracted. In some embodiments, due to noisy margin ink heatmaps, preprocessing may be applied to the contour. For example, the contour may be estimated as a polygon and partitioned from a single large list of points to a set of smaller lists of points, where each list in the set represents a contiguous “segment” of the contour. The contour points may be determined to be margin or non-margin points. For example, contour segments with fewer than 10 points may be assigned as non-margin to prevent noise and contour segments with more than 25% of its points contained within regions of the margin heatmap may be assigned as margin points. Objects in the cancer heatmap may be assigned to distinct sections of tissue based on intersection. Within each tissue section, the minimum distance between cancer pixels and margin pixels may be identified and used as the margin distance for that tissue section. When multiple tissue sections are identified, across all tissue sections, the margin distances may be aggregated, and outliers (e.g., more than 1.01 standard deviations below or above the mean margin distance) may be removed. Then, the minimum distance is selected from the aggregated distances and used as the margin distance for the histopathology image.

Using the techniques developed by the inventors, the margin assessment for detected tumors was accepted by pathologists without any alterations in about 50% of cases. The results of this testing are shown in Table 3 below. The results indicate that the margin assessment performed by histopathology image analysis systems may improve the review by pathologists and therefore improve pathologist efficiency.

TABLE 3

| | Pathologist evaluation | | | | | |
|---|---|---|---|---|---|---|
| AI Model | | Does the | Would the | | | |
| Result | | slide contain | pathologist | | | |
| Did the AI | | a section | accept the AI | | | |
| provide a | Does the | with both | assessment as- | | | |
| margin | slide contain | cancer and | is for a | AI Model | | |
| assessment? | cancer? | margin ink? | synoptic report? | Pass/Fail? | Results | |
| Yes | Yes | Yes | Yes | Pass | 86 | (29%) |
| | | | No | Fail | 109 | (36%) |
| | | No | N/A | Fail | 4 | (1%) |
| | No | N/A | | Fail | 23 | (8%) |
| No | Yes | Yes | N/A | Fail | 13 | (4%) |
| | | No | | Pass | 25 | (8%) |
| | No | N/A | | Pass | 40 | (13%) |

| | | |
|---|---|---|
| Total | 300 | (100%) |

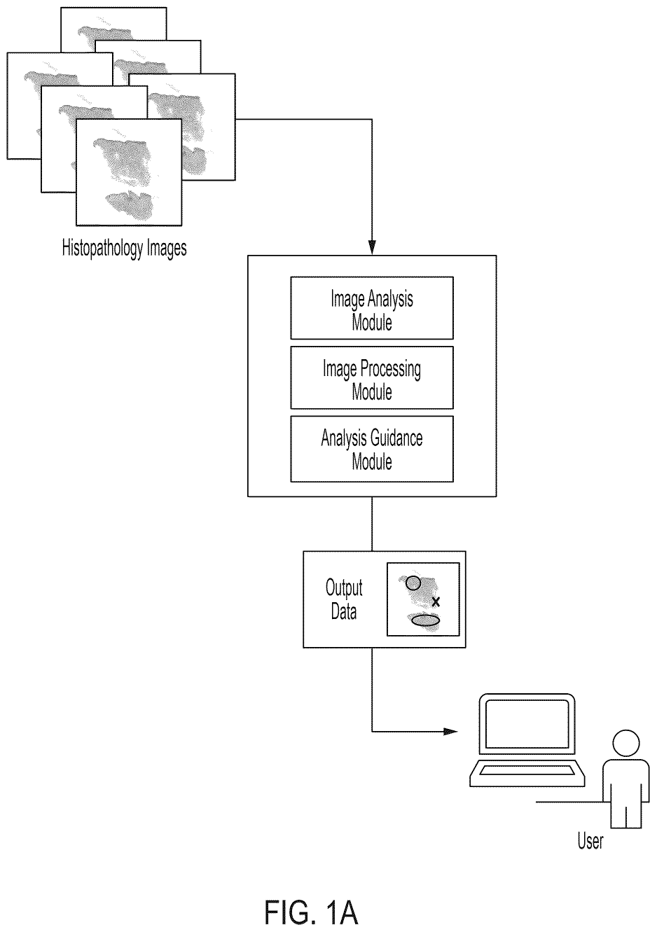

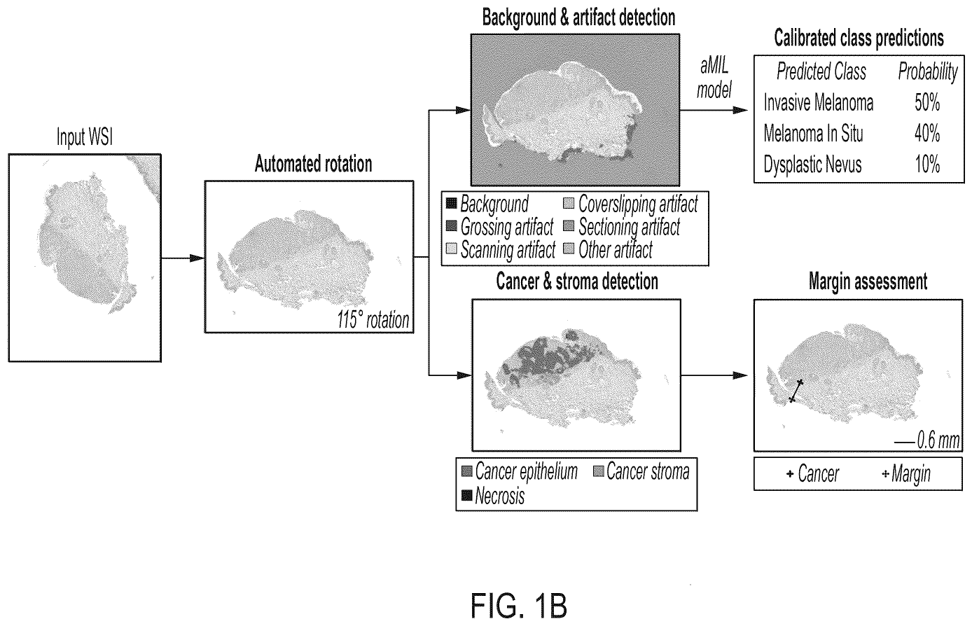

FIG. 1A is an example environment to which the technology described herein may be deployed. As shown, histopathology images are obtained, for example from a lab environment and passed to a histopathology image analysis system. The analysis system may include one or more computer hardware processors for performing the functions described herein. The system includes an image analysis module, an image processing module, and an analysis guidance module. The image analysis module may perform one or more actions to analyze a histopathology image, as described herein. In some embodiments, the image analysis module may determine probabilities of one or more diagnostic entities being present in a histopathology image, such as by using a trained machine learning model, as described herein. In some embodiments, the image analysis module may determine a mitotic rate, and/or a margin assessment from a histopathology image, as described herein. The image analysis module may include one or more trained machine learning models and/or computer models for performing the functions described herein. The image processing module may perform one or more actions to generate a modified image to output. In some embodiments, the image processing module may reorient a histopathology image, apply one or more fields of interest to a histopathology image, apply one or more overlays to a histopathology image, and/or determine a lesion depth within a histopathology image, as described herein. The analysis guidance module may determine data to output to a user of the histopathology system. In some embodiments, the analysis guidance module may determine one or more diagnostic entities to output which are most likely to be included in a histopathology image. The diagnostic entities and size of the output set may be determined based on the probabilities determined by the diagnostic entity identification module, as described herein. The output set may be determined using a conformal prediction technique. In some embodiments, the analysis guidance module may determine one or more recommended stains to output to a user. The output data is then provided to a user device for review by a user, e.g., a pathologist. The output data may include a modified image and information related to the output set.

FIG. 1B is an example process which may be performed by a histopathology image analysis system. The process includes automated rotation of a histopathology image, followed by background and artifact detection and application of a machine learning model to determine an output set containing calibrated class predictions of diagnostic entities present in the histopathology image. The process additionally includes cancer and stroma detection followed by margin assessment if cancer and margin ink are detected. The regions of cancer and stroma may be detected using a ML model as described herein.

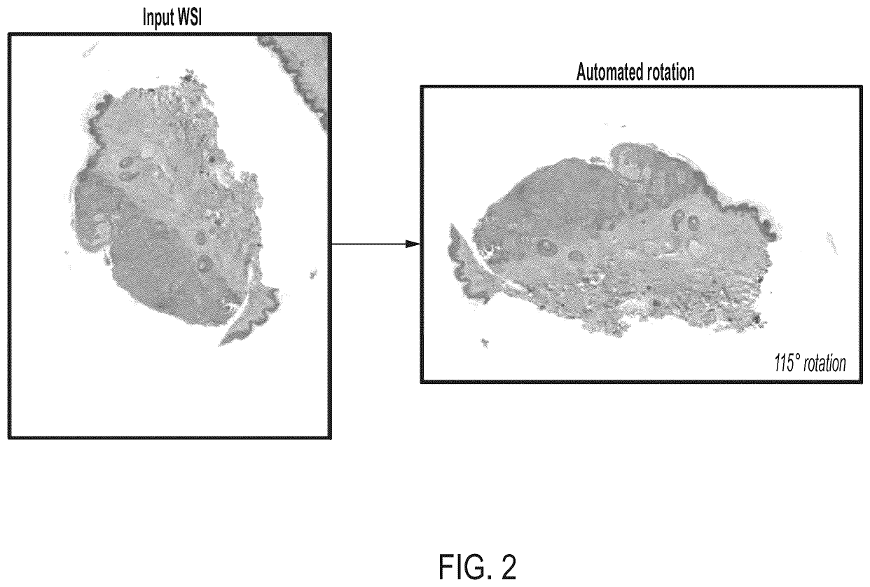

FIG. 2 illustrates a slide depicting a skin biopsy prior to and following application of automated orientation techniques, according to some embodiments of the technology described herein. Upon receiving a slide, the method described herein identifies a target rotation (115° in this example). When the target rotation is applied to the input slide, the slide is reoriented to be displayed in accordance with the pathologist's preference (e.g., with a particular tissue being displayed on the north portion of the slide). Identifying the target rotation and applying the target rotation to the input slides are steps that may be performed without having to require user intervention. As described herein, this automatic rotation reduces the time and input needed from pathologists when reviewing individual slides.

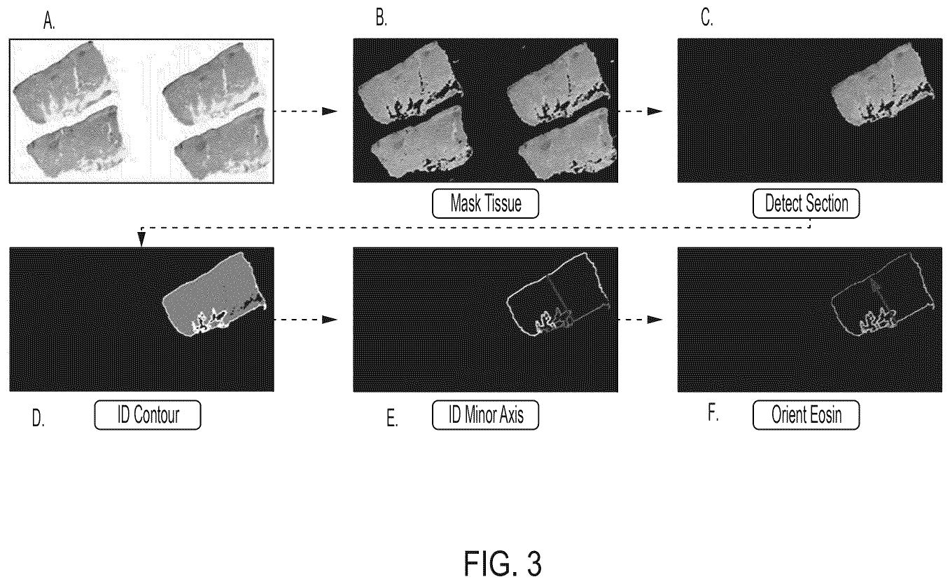

FIG. 3 illustrates the steps of a representative method for performing automatic orientation of a slide, in accordance with some embodiments. In this example, the slide represents a skin specimen stained with Hematoxylin and Eosin (H&E) Stain. As described in detail below, in this example, the method identifies a target tissue (e.g., epidermis), and reorients the slide so that the epidermis is displayed facing “north” (the upper portion of the image).

The method described in connection with FIG. 3 may be implemented using one or more processors coupled to a memory storing instructions that, when executed, cause the one or more processors to execute the method. At step A, the processor(s) receive a slide (a histopathology image) depicting tissues obtained from a biopsy (a skin biopsy in this example).

At step B, the processor(s) isolate specific tissues of the slide from the background using a mask. The mask may additionally remove small objects from the image to improve clarity. The mask may be implemented for example as a machine learning model trained to identify particular types of tissues. Alternatively, techniques such as Otsu masking, triangle masking, minimum error thresholding, Li's thresholding method, Yen's thresholding method, or other automatic thresholding methods may be used to mask the target tissue from the histopathology image.

At step C, the processor(s) further isolates the target tissue (e.g., epidermis) by deleting from the slide tissues that are of less interest. The target tissue may be first determined by performing a connected components analysis on the masked image to isolate individual tissue samples within the image. One or more tissue samples may be isolated as the target tissue,

At step D, the processor(s) identify the contour of the target tissues, for example using a machine learning model trained to identify contours of particular types of tissues. The contour may be the border of the isolated target tissue, having a predetermined width, as described herein.

At step E, the processor(s) identify an axis of symmetry associated with the contour of the target tissue. In the example of FIG. 3, the minor axis is identified, although other embodiments may identify the major axis. The axis may be determined based on the shape of the contour, for example based on the smallest ellipse bounding the contour.

The processor(s) identify the orientation of the axis, which is used as a proxy to identify the orientation of the target tissue. Whether to identify the minor axis, the major axis or other axes of symmetry may depend on the expected shape of the tissue. Skin specimens tend to be generally rectangular in shape, making them particularly suitable to use the minor axis as a proxy of the orientation of the target tissue. The orientation of the contour may be determined using information from the histopathology image, for example the coloring of the target tissue. Different stains result in different tissue colors. For example, in an H&E stain of a skin sample, the epidermis appears darker and more purple than surrounding tissue. Based on this coloring, the contour may be mapped to the minor axis of a tissue sample and used to determine the half of the axis which is darker and/or more purple (and therefore indicative of the half of the axis facing the epidermis).

At step F, the processor(s) identify the rotation axis that, when applied to the target tissue, causes the target tissue to be displayed in accordance with the preferred orientation. For skin samples, this is with the epidermis facing the top of the image, therefore the rotation to be applied to the image is the rotation which positions the minor axis vertically with the half associated with the epidermis facing up. Lastly, the processor(s) apply the orientation to the target tissue and cause a display device to display the reoriented target tissue. In the example of FIG. 3, the reoriented slide is displayed so the epidermis faces north. In the context of GI slides, a slide may be reoriented to display the outer surface of the GI tract on the right hand side.

When multiple target tissues are present, the above-described steps may be performed for each of the target tissues in the image to determine the rotations to be applied. In some examples, the individual tissues may be rotated according to the determined rotations. In some examples, the entire histopathology image is rotated based on a weighted average of the rotations to be applied to the individual tissue samples, weighted by the areas of the tissue samples.

In one particular use case, the inventors have applied automated orientation to skin specimens leveraging major and minor axis detection and staining intensity to place the epidermis horizontally at the top of a whole slide image (WSI). Neoplastic lesions were identified using a model trained using pathologist annotations of cancer and stroma regions. The model for AI-assisted diagnosis and case prioritization was trained on a diverse set of hematoxylin and eosin (H&E)-stained WSIs (N=11543) using a pathology foundation model with additive multiple instance learning (MIL) for classification. Slide orientation was evaluated on H&E-stained skin WSIs (N=274) obtained from three independent anatomic pathology laboratories. AI-assisted and manual pathologist diagnoses were compared (N=2214 WSI). A simulation to sort cases with and without automated triaging was performed, incorporating ground truth and model true and false positive rates, and metrics were assessed for basal cell carcinoma (BCC), squamous cell carcinoma (SCC), and melanoma (MEL).

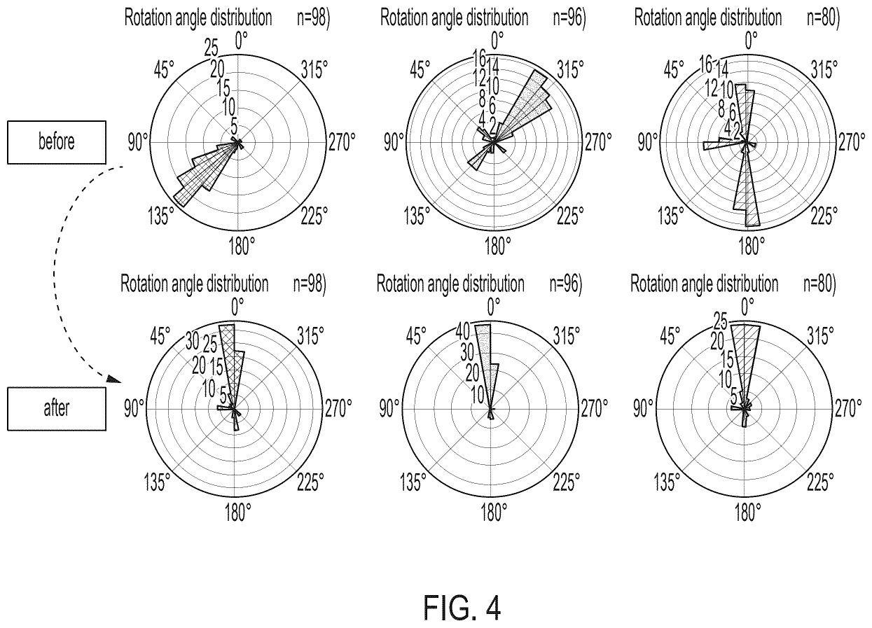

Automated orientation has been observed to decrease the fraction of WSI needing manual rotation from 90% (N=246/274) to 30% (N=83/274) (FIG. 4). AI-assisted diagnosis was accurate for MEL and MEL in situ (TPR=0.91, FPR=0.1), BCC (TPR=0.92, FPR=0.03), and SCC and SCC in situ (TPR=0.81, FPR=0.05). Simulated case prioritization revealed higher priority and earlier review for slides predicted to contain lesions: use of the model for triage increased the median percent of high-priority cases reviewed in the first half of the day from 50% at baseline to 73% (BCC), 69% (SCC), and 73% (MEL). FIG. 4 shows the above results. The top row of charts depicts the angle rotations applied by pathologists to unrotated histopathology images, and the bottom row shows the angle rotations applied by pathologists to histopathology images which underwent automatic rotation.

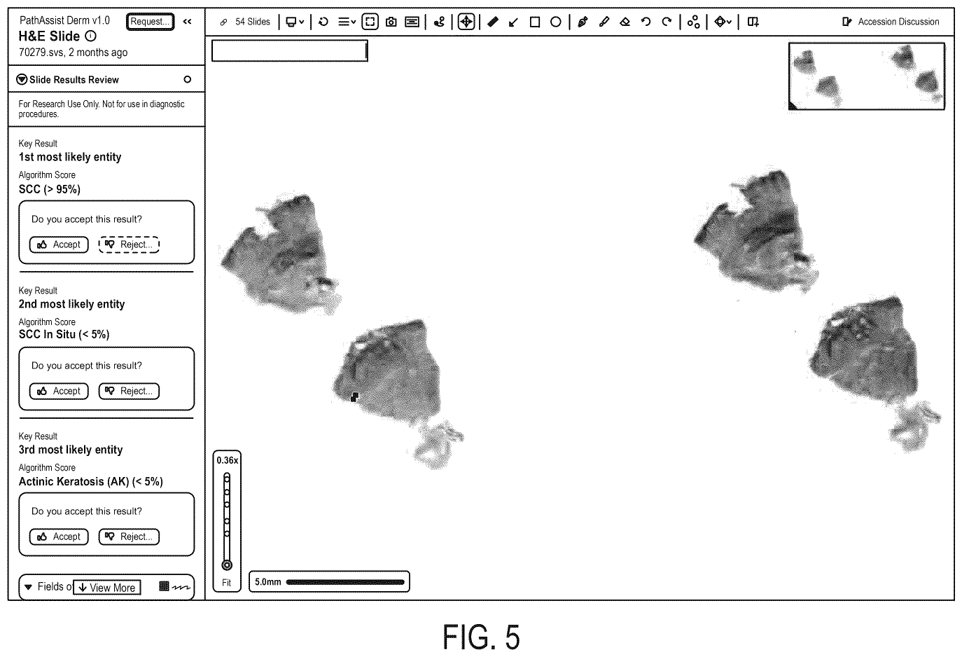

FIG. 5 is an example of data which may be provided on a user interface of a user device during a review. The data includes indications of the most likely entities or conditions within the specimen, with the confidence scores for the presence of these diagnostic entities. These likelihoods may be determined as described herein, for example using a machine learning model and by determining an output set using conformal prediction. On the right the associated histopathology image is shown, with controls for the user to analyze the image. The user interface additionally includes options for the reviewer to accept or reject the predicted diagnostic entities. When an entity is accepted, it may be associated with the histopathology image (e.g., in storage of the histopathology image analysis system). When an entity is rejected, the entity may be removed from the list of entities displayed on the left and one or more additional entities may be displayed for review.

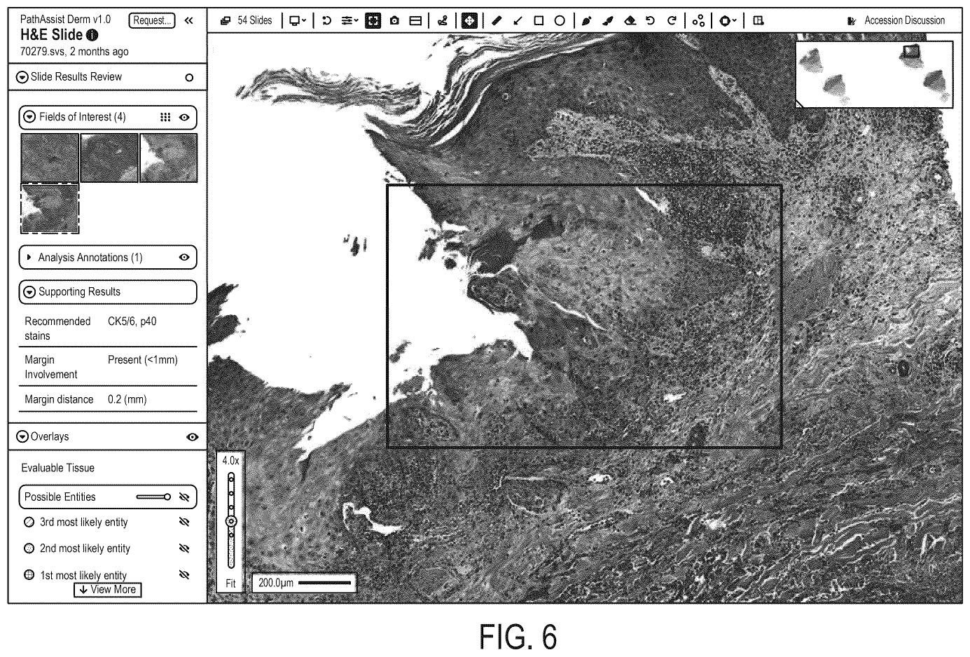

FIG. 6 is an example of a field of interest which may be provided on a user interface during a review. As shown, the menu on the left includes options to select a field of interest from four fields of interest associated with a selected diagnostic entity. Also shown are toggles for overlays which, in FIG. 6, are not enabled and options to select other diagnostic entities for review. The right part of the interface shows the field of interest as a box superimposed on the histopathology image and controls for the user to analyze the image. The field of interest may additionally be enlarged to facilitate review by the pathologist. The fields of interest may be determined as described herein, for example by using a machine learning model and/or based on the probabilities of diagnostic entities being present in the histopathology image determined by a machine learning model. For example, the fields of interest may be determined based on the portions of the histopathology image with the largest areas of substance and/or features of interest associated corresponding to and/or indicative of diagnostic entities.

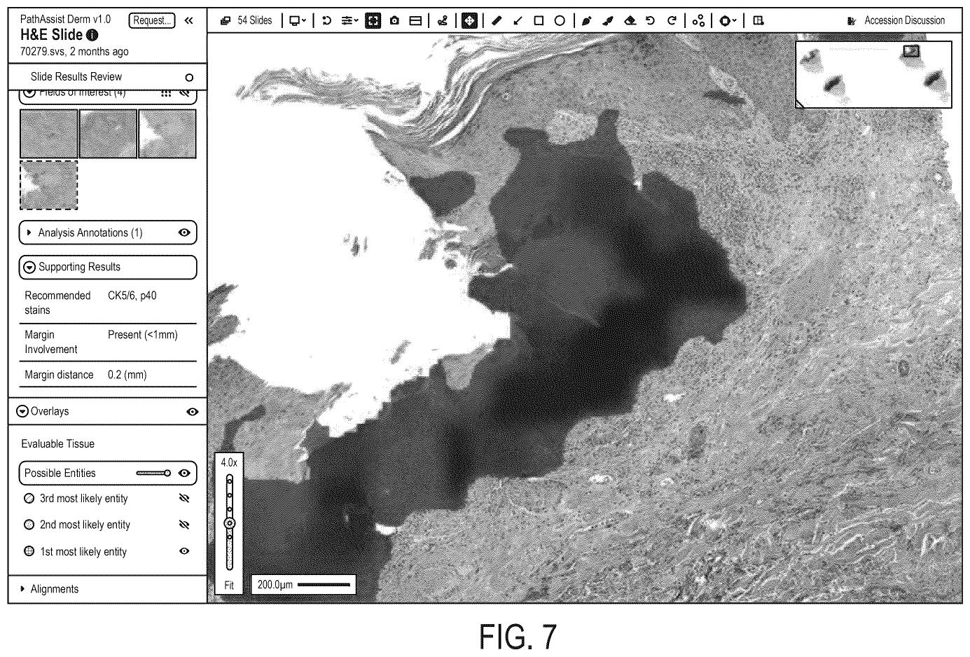

FIG. 7 is an example of an overlay which may be provided on a user interface during a review. As shown, the menu on the left of the display provides inputs for a user to select a location for a particular overlay associated with a current diagnostic entity. Also shown are options for a user to select other overlays associated with other diagnostic entities. As shown, the other diagnostic entities may have overlays in different colors. On the right side of the interface is the histopathology image with the overlay applied. The overlay is shown as the dark sharded region over the specimen in the histopathology image. The overlays may be determined as described herein, for example by using a machine learning model and/or based on the probabilities of diagnostic entities being present in the histopathology image determined by a machine learning model. For example, the overlays may correspond to portions of the histopathology image corresponding to and/or indicative of a particular diagnostic entity, for example portions of the image including particular features or substances of interest for the diagnostic entities.

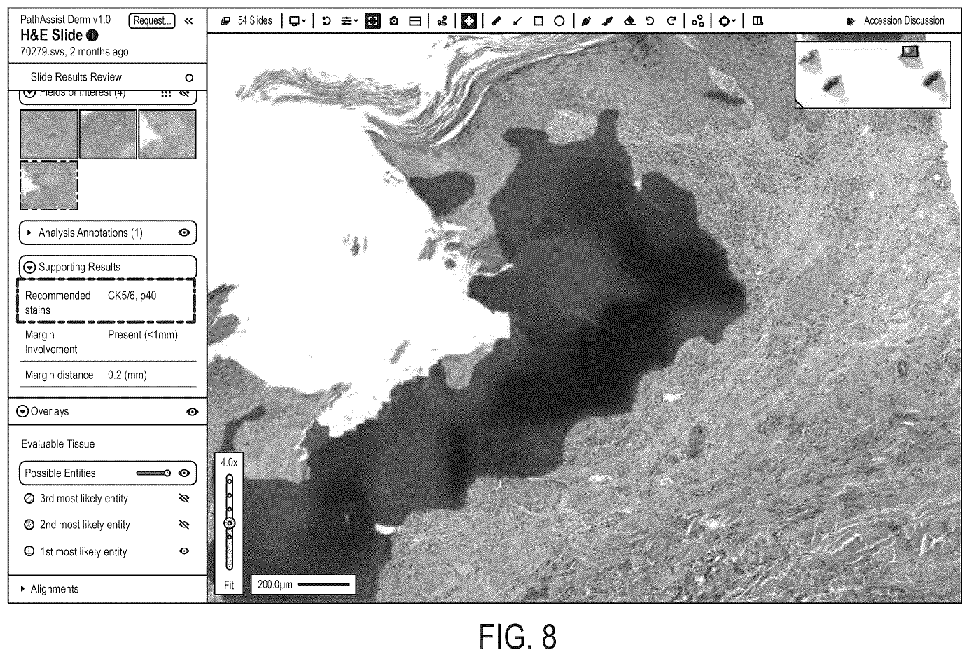

FIG. 8 is an example of a user interface of a histopathology analysis system including recommended stains. As shown, a squamous cell carcinoma is the most likely diagnostic entity present in the histopathology image at the right of the interface. This may be indicated in an output set of diagnostic entities. The recommended stains for this specimen are CK5/6 and p40 stains, which may assist in diagnosing this specimen following future imaging, as described herein. The recommended stains may be determined as described herein, for example based on known stains which are useful in confirming the presence of the diagnostic entity. Stains may additionally be recommended based on stains known to distinguish between multiple diagnostic entities contained within the output set. For example, a particular stain may be useful in differentiating between a squamous cell carcinoma and Actinic Keratosis, which may otherwise be difficult to distinguish.

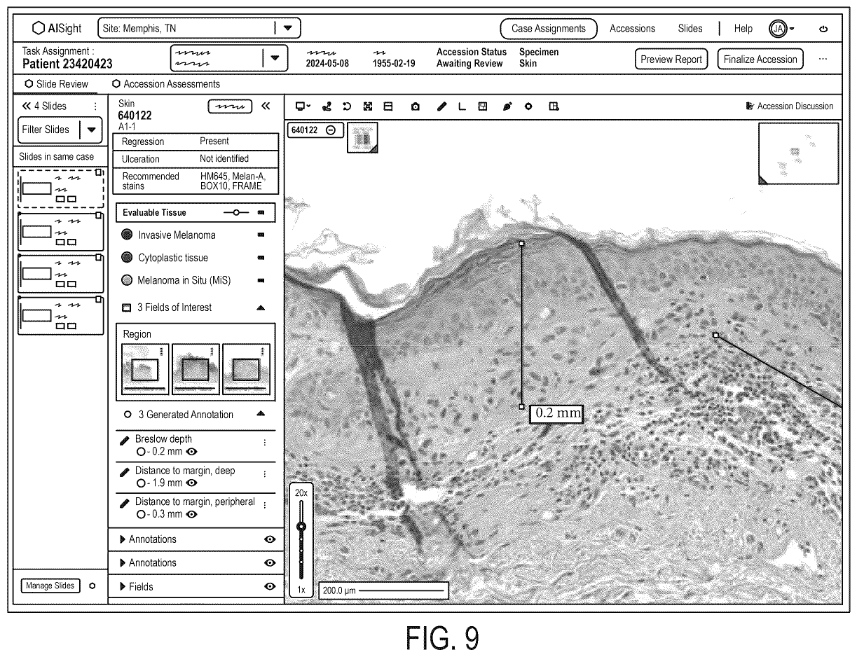

FIG. 9 is an example of a user interface of a histopathology analysis system including a lesion depth measurement. The lesion depth measurement is shown on the histopathology image at the right side of the interface. As shown, the lesion depth is 0.2 mm. In some embodiments, the user interface may allow a user to select points on a histopathology image to measure. The lesion depth may be determined as described herein. The user interface may additionally provide tools for a pathologist to determine lesion depth (e.g., via a click and drag tool). As shown, in the menu on the left of the interface, three different measurements have been determined by the analysis system and presented to the user, including the Breslow depth, the deep distance to margin and the peripheral distance to margin. In some embodiments, greater or fewer measurements may be provided.

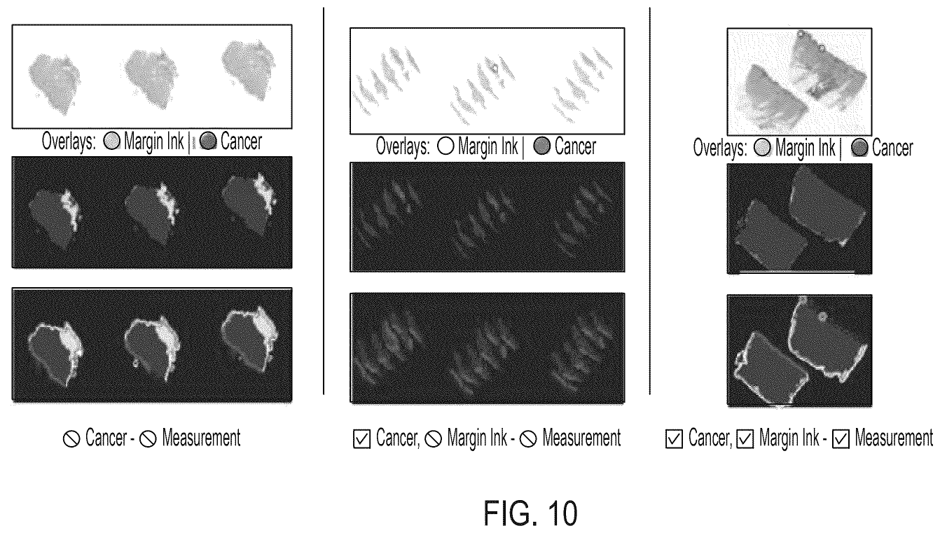

FIG. 10 is an example of determining whether to perform a margin assessment on a histopathology image. The process shown in FIG. 10 may be performed by a histopathology image analysis system as described herein. As shown, in the images on the left of FIG. 10, no cancer is detected in the histopathology image, therefore a margin assessment is not performed. In the center image, cancer is detected, but margin ink is not detected, therefore a margin assessment is not performed. Finally, on the right images, margin ink and cancer are detected therefore a margin assessment is performed, as shown in the bottom image. The margin assessment may be performed as described herein. For example, the margin assessment may be performed using heatmaps determined by and/or from the ML model. Such heatmaps are shown in FIG. 10 for cancer and margin ink. The heatmaps may undergo processing, such as an opening operation and dilation, followed by a connected components analysis to isolate the tissues within the images. The contours may be determined and smoothed and used to determine whether they are margin or non-margin. Within each tissue section, the minimum distance between cancer pixels and margin pixels may be identified and used as the margin distance for that tissue section. When multiple tissue sections are identified, across all tissue sections, the margin distances may be aggregated, and outliers may be removed. The minimum distance may be used as the margin distance for the histopathology image.

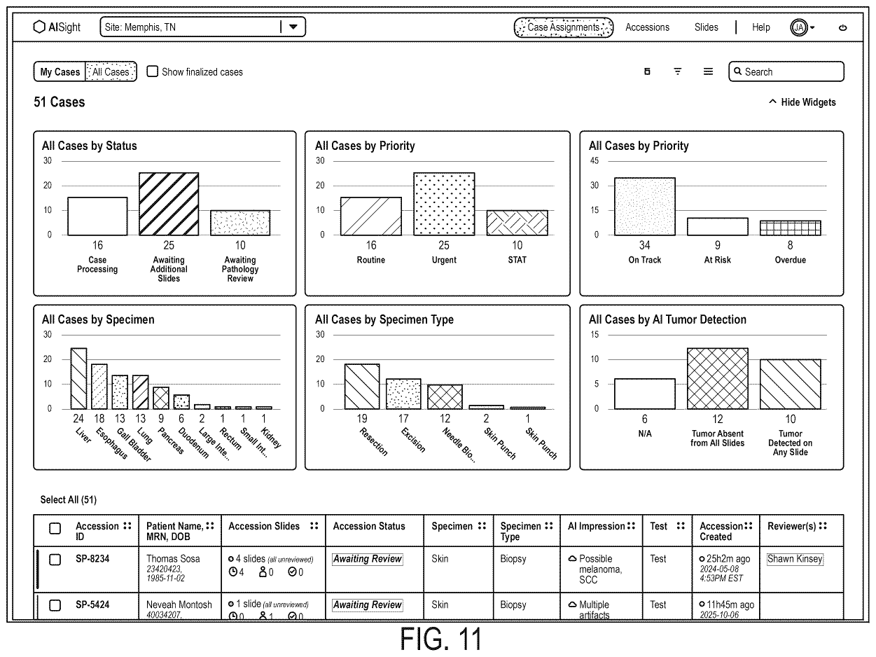

FIG. 11 is an example of a workload display of a user interface of a histopathology image analysis system. The display of FIG. 11 may be available to a user through a page of the user interface. As shown, the workload display includes data related to histopathology images which may be ready for review. The displayed workload may be for an individual user or a group of users. As shown, the workload display includes a view of case status, including case processing, awaiting additional slides, and awaiting pathology review. The display includes a view of the priority of cases including routine, urgent and stat. In some embodiments, case priority may be determined based on diagnostic entities determined to be present in the specimen, such as determined by the histopathology image analysis system and/or an output set of cases. The display includes a view of the case progress including on track cases, at risk cases and overdue cases. The display include a view of specimen source. The display includes a view of specimen type including resection, excision, needle biopsy, skin punches. The display includes a view of AI tumor detection, including N/A, tumor absent from all slides, and tumor detected on any slide. In some embodiments, the AI tumor detection may be performed by the histopathology image analysis system, for example using one or more machine learning models, as described herein. The display includes a list view of histopathology cases, the list view showing an ID for a case, a patient name and date of birth, an indications of the slides present for the case, a status for the case, a specimen source for the case, a specimen type for the case, an AI impression for the case, whether a test is required for the case, an accession created time for the case and a reviewer for the case. In some embodiments, greater or fewer data may be shown for a case. In some embodiments, the AI impression may be determined by one or more machine learning models of the histopathology image analysis system, for example based on probabilities of diagnostic entities being present and/or an output set of diagnostic entities. A pathologist may review the workload display to prioritize cases for review, as described herein. In some embodiments, the user interface may provide users with the option to filter cases, for example, by status, priority, progress, specimen, specimen type, and/or tumor detection, among other possible filters.

It should be noted that the techniques described herein can be applied to specimens other than skin (including for example gastrointestinal (GI) tract, liver, bone marrow, kidney, muscles, lung, etc.). Additionally, the techniques described herein can be applied to specimens stained using stains other than H&E, including for example Periodic Acid-Schiff (PAS) Stain, Masson's Trichrome Stain, Silver Stains, Immunohistochemistry (IHC) Stains, etc. The stain should be selected, among other factors, to isolate a particular tissue that the methods described herein can recognize and use to orient a slide.

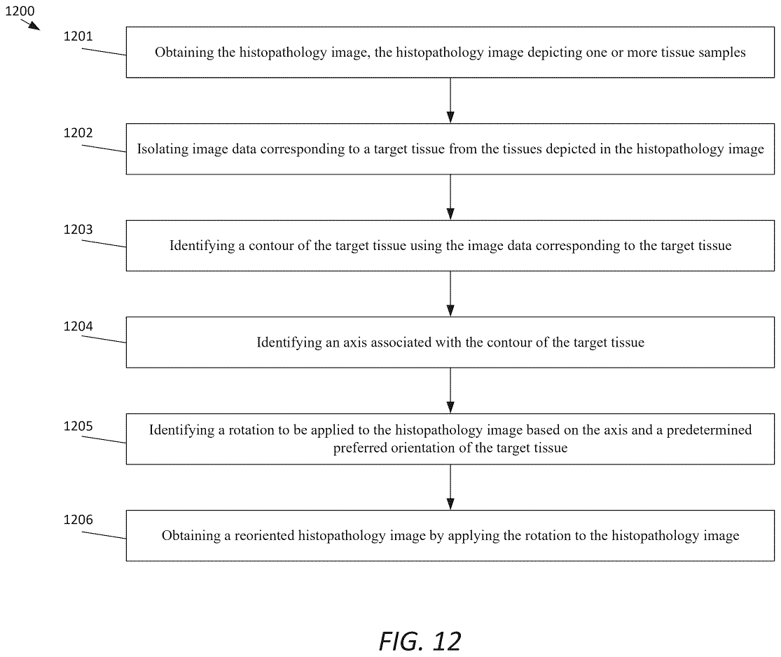

FIG. 12 is an example process for preparing a histopathology image for analysis, according to some embodiments of the technology described herein. Process 1200 may be performed by a histopathology image analysis system, as described herein.

Process 1200 begins with act 1201, in which a histopathology image depicting one or more tissue samples is obtained. The histopathology may be a digital image and may have any suitable format, as described herein. The image may be obtained from a database, a scanner or any suitable source. The one or more tissue samples may be obtained from a biopsy, as described herein. The one or more tissue samples may be processed as described herein, such as by having one or more stains applied.

The process proceeds to act 1202, in which image data corresponding to a target tissue is isolated from the histopathology image. The target data may correspond to a particular type of tissue, as described herein. The target tissue may be isolated using a masking process, such as an automatic thresholding process. After masking a connected components analysis may be performed to generate components, representing the tissue segments within the histopathology image. The image data corresponding to the locations of these components may be isolated as the target tissues.

The process then proceeds to act 1203, in which a contour of the target tissue is identified using the image data corresponding to the target tissue. The contour may be the border of the target tissue having a predetermined width, and may be determined as described herein, such as through edge detection.

The process then proceeds to act 1204, in which an axis associated with the target tissue is identified. The axis may be identified based on the shape of the target tissue. For example, an ellipse bounding the contour of the target tissue may be identified, and the axis may be the major or minor axis of the ellipse. In some embodiments, the axis may be determined based on the type of target tissue, as specific target tissues may have known and/or expected shapes. For example, skin samples are often elongated parallel to a surface of the skin, therefore the minor axis is approximately perpendicular to the surface of the skin or epidermis.

The process then proceeds to act 1205, in which the rotation to be applied to the histopathology image is identified based on the axis and a predetermined orientation of the target tissue. The predetermined orientation of the target tissue may correspond to the type of the target tissue. For example, skin samples may have a preferred orientation with the epidermis facing the top of the image. The rotation to be applied based on one or more image features or artifacts within the histopathology image. For example, pixel colors may be used, as described herein, to identify the epidermis in skin samples and identify the half of the axis in which the target tissue is located. The rotation may then be determined as the rotation that orients the axis vertically with the epidermis facing the top of the histopathology image.

The process then proceeds to act 1206, in which the reoriented histopathology image is obtained by applying the rotation to the histopathology image. The image may be rotated by the identified rotation. After generating the reoriented image, the image may be displayed on one or more user interfaces, such as a user interface of a histopathology image analysis system as described herein.

In some embodiments, multiple target tissues are identified in a histopathology image and the rotation to be applied to the histopathology image are determined based on the multiple target tissues. For example, in act 1202, multiple target tissues may be identified and acts 1203-1205 may be performed for each of the target tissues. The rotations determined for each of the target tissues may be used to determine the rotation to be applied to the histopathology image. For example, a weighted average rotation may be determined, weighted by the areas of the target tissues. This weighted average rotation may then be applied to the histopathology image.

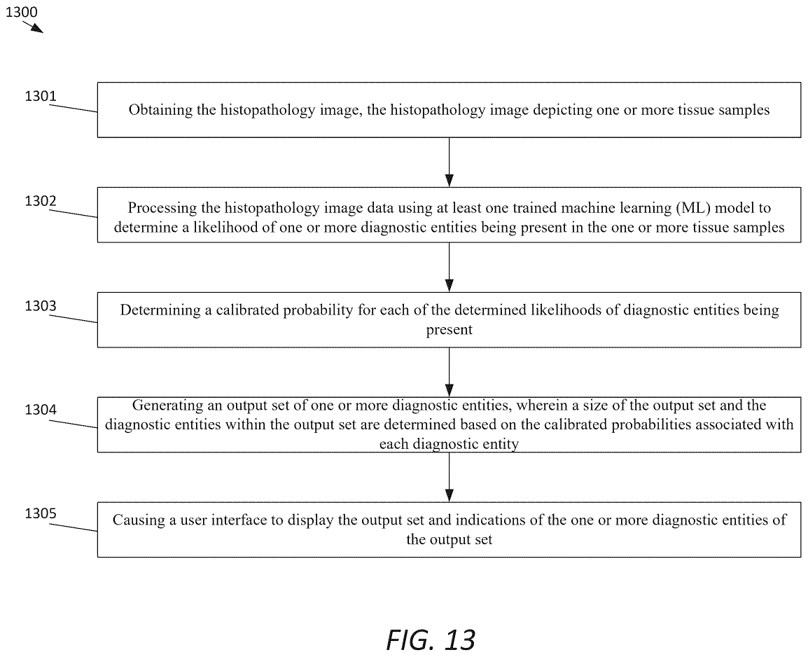

FIG. 13 is an example process for analyzing a histopathology image, according to some embodiments of the technology described herein. Process 1300 may be performed by a histopathology image analysis system, as described herein.

Process 1300 begins with act 1301, in which a histopathology image depicting one or more tissue samples is obtained. The histopathology may be a digital image and may have any suitable format, as described herein. The image may be obtained from a database, a scanner or any suitable source. The one or more tissue samples may be obtained from a biopsy, as described herein. The one or more tissue samples may be processed as described herein, such as by having one or more stains applied.

The process then proceeds to act 1302, in which the histopathology image data is processed using at least one trained ML model to determine a likelihood of one or more diagnostic entities being present in the histopathology image. The ML model may be an ML model as described herein such as a foundational model with a backbone and one or more adaptation heads. The ML model may output a set of entities and associated likelihoods of being present in the histopathology image. The ML model may additionally output locations of image features or artifacts indicative of or corresponding to the entities and/or heatmaps indicating the locations of the features or artifacts.

The process then proceeds to act 1303, in which a calibrated probability is determined for each of the diagnostic entities being present. The calibrated probability may be determined from the outputs of the ML model and/or the ML model may be configured to output calibrated probabilities. The calibrated probabilities may be determined based on a holdout set of labeled histopathology images, and the confidence of the ML model may be compared to the performance of the model for each of the entities for which it is configured to predict.

The process then proceeds to act 1304, in which an output set of one or more diagnostic entities is generated using the calibrated probabilities. The size of the output set and the entities within the output set may be determined using the calibrated probabilities. The output set may be determined using a conformal prediction technique, as described herein, for example using adaptive prediction sets or another technique described herein.

The process then proceeds to act 1305, in which a user interface is caused to display the output set and indication of the one or more diagnostic entities of the output set. The user interface may display the histopathology image with the indications of the diagnostic entities labeled, for example as fields of interest and/or overlays on the histopathology image.

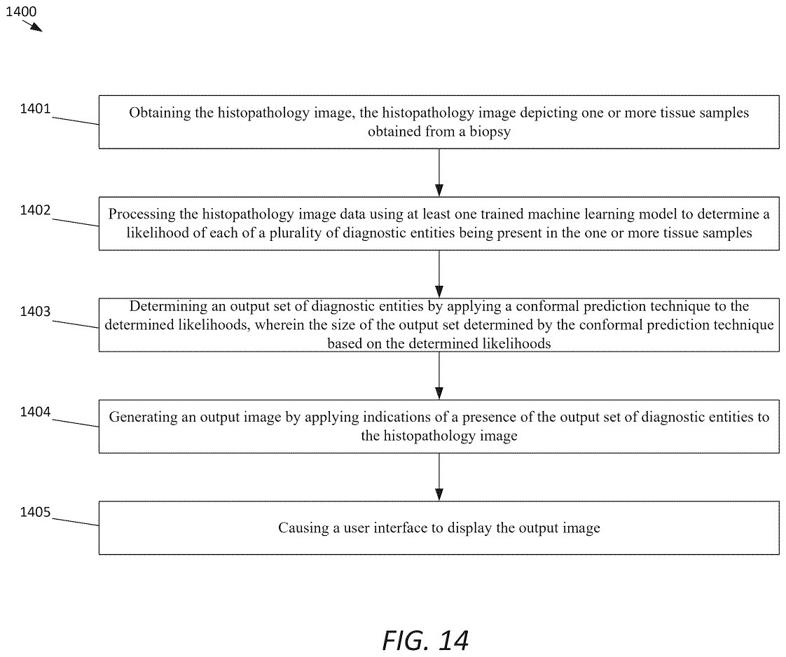

FIG. 14 is an example process for analyzing a histopathology image, according to some embodiments of the technology described herein. Process 1400 may be performed by a histopathology image analysis system, as described herein.

Process 1400 begins with act 1401, in which a histopathology image depicting one or more tissue samples is obtained. The histopathology may be a digital image and may have any suitable format, as described herein. The image may be obtained from a database, a scanner or any suitable source. The one or more tissue samples may be obtained from a biopsy, as described herein. The one or more tissue samples may be processed as described herein, such as by having one or more stains applied.

The process then proceeds to act 1402, in which the histopathology image data is processed using at least one trained ML model to determine a likelihood of one or more diagnostic entities being present in the histopathology image. The ML model may be an ML model as described herein such as a foundational model with a backbone and one or more adaptation heads. The ML model may output a set of entities and associated likelihoods of being present in the histopathology image. The ML model may additionally output locations of image features or artifacts indicative of or corresponding to the entities and/or heatmaps indicating the locations of the features or artifacts. In some embodiments, the outputs of the ML model may be calibrated, as described herein.

The process then proceeds to act 1403, in which an output set of diagnostic entities are determined by applying a conformal prediction technique to the determined likelihoods. The size of the output set is determined by the conformal prediction technique based on the determined likelihoods. The conformal prediction techniques may include those described herein, for example using adaptive prediction sets.

The process then proceeds to act 1404 in which an output image is generated by applying indications of a presence of the output set of diagnostic entities to the histopathology image. For example, one or more overlays and/or fields of interest may be applied to the histopathology image. The output image may additionally be oriented, as described herein, such as by applying a rotation to the image determined based on a target tissue within the image.

The process then proceeds to act 1405 in which a user interface is caused to display the output image.

It should be appreciated that one or more embodiments may be practiced alone or in combination with other features, systems, processes, and interfaces of one or more systems. Some exemplary systems are described in U.S. Pat. Nos. 12,073,948 and 11,908,139 which are incorporated by reference by their entirety.

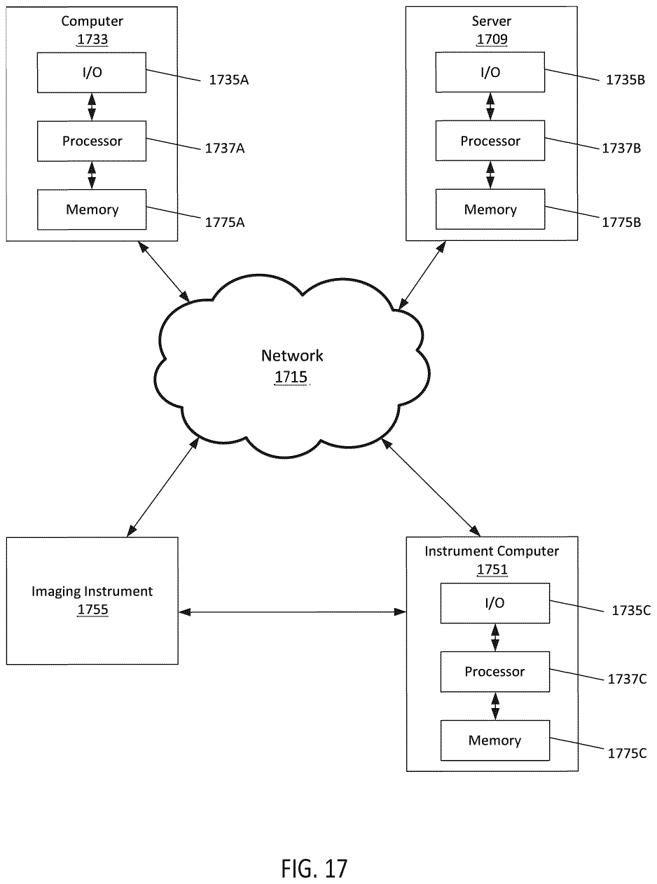

FIG. 17 shows a block diagram of a computer system on which various embodiments of the technology described herein may be practiced. The system includes at least one computer 1733. Optionally, the system may further include one or more of a server computer 1709 and an imaging instrument 1755 (e.g., one of the instruments described above which may be used in capturing pathology images), which may be coupled to an instrument computer 1751. Each computer in the system, computer 1733, server 1709 and instrument computer 1751, includes a respective processor 1737A, 1737B and 1737C coupled to a respective tangible, non-transitory memory device 1775A, 1775B, and 1775C and at least one respective input/output device 1735A, 1735B and 1735C. Thus, the system includes at least one processor coupled to a memory subsystem (e.g., a memory device or collection of memory devices). In some embodiments, the memory devices, 1775A, 1775B, 1775C, may be separate memory devices. In some embodiments, the memory devices, 1775A, 1775B, 1775C, may be a single memory device. The components (e.g., computer, server, instrument computer, and imaging instrument) may be in communication over a network 1715 that may be wired or wireless and wherein the components may be remotely located or located in close proximity to each other. Using those components, the system is operable to receive or obtain image data such as whole-slide images, pathology images, histology images, or tissue images and annotation and score data as well as test sample images generated by the imaging instrument or otherwise obtained. In certain embodiments, the system uses the memory to store the received data as well as the model data which may be trained and otherwise operated by the processor.

In some embodiments, some or all of the system is implemented in a cloud-based architecture. The cloud-based architecture may offer on-demand access to a shared pool of configurable computing resources (e.g. processors, graphics processors, memory, disk storage, network bandwidth, and other suitable resources). A processor in the cloud-based architecture may be operable to receive or obtain training data such as whole-slide images, pathology images, histology images, or tissue images and annotation and score data as well as test sample images generated by the imaging instrument or otherwise obtained. A memory in the cloud-based architecture may store the received data as well as the model data which may be trained and otherwise operated by the processor. In some embodiments, the cloud-based architecture may provide a graphics processor for training the model in a faster and more efficient manner compared to a conventional processor.

Processor refers to any device or system of devices that performs processing operations. A processor will generally include a chip, such as a single core or multi-core chip (e.g., 12 cores), to provide a central processing unit (CPU). In certain embodiments, a processor may be a graphics processing unit (GPU) such as a Nvidia Tesla K80 graphics card from NVIDIA Corporation (Santa Clara, CA). A processor may be provided by a chip from Intel or AMD. A processor may be any suitable processor such as the microprocessor sold under the trademark XEON E5-2620 v3 by Intel (Santa Clara, CA) or the microprocessor sold under the trademark OPTERON 6200 by AMD (Sunnyvale, CA). Computer systems may include multiple processors including CPUs and/or GPUs that may perform different steps of the described methods. The memory subsystem may contain one or any combination of memory devices (e.g., memory devices 1775A, 1775B, 1775C). A memory device is a mechanical device that stores data or instructions in a machine-readable format. Memory may include one or more sets of instructions (e.g., software) which, when executed by one or more of the processors of the disclosed computers can accomplish some or all of the methods or functions described herein. Each computer may include a non-transitory memory device such as a solid state drive, flash drive, disk drive, hard drive, subscriber identity module (SIM) card, secure digital card (SD card), micro SD card, or solid state drive (SSD), optical and magnetic media, others, or a combination thereof. Using the described components, the system is operable to produce a report and provide the report to a user via an input/output device. An input/output device is a mechanism or system for transferring data into or out of a computer. Exemplary input/output devices include a video display unit (e.g., a liquid crystal display (LCD) or a cathode ray tube (CRT)), a printer, an alphanumeric input device (e.g., a keyboard), a cursor control device (e.g., a mouse), a disk drive unit, a speaker, a touchscreen, an accelerometer, a microphone, a cellular radio frequency antenna, and a network interface device, which can be, for example, a network interface card (NIC), Wi-Fi card, or cellular modem.

It is to be appreciated that embodiments of the methods and apparatuses discussed herein are not limited in application to the details of construction and the arrangement of components set forth in the present disclosure or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, any embodiment disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, or ordinary meanings of the defined terms.

The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

As referred to herein, the term “in response to” may refer to initiated as a result of or caused by. In a first example, a first action being performed in response to a second action may include interstitial steps between the first action and the second action. In a second example, a first action being performed in response to a second action may not include interstitial steps between the first action and the second action.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having the same name (but for use of the ordinal term).

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the systems and methods described herein. Accordingly, the foregoing description and drawings are by way of example only.

What is claimed is:

1. A method for analyzing a histopathology image, the method comprising:
using at least one computer hardware processor to perform:
obtaining the histopathology image, the image containing histopathology data of one or more tissue samples;
processing the histopathology image data using at least one trained machine learning (ML) model to determine a likelihood of one or more diagnostic entities being present in the one or more tissue samples;
determining a calibrated probability for each of the determined likelihoods of diagnostic entities being present;
generating an output set of one or more diagnostic entities, wherein a size of the output set and the diagnostic entities within the output set are determined based on the calibrated probabilities associated with each diagnostic entity; and
causing a user interface to display the output set and indications of the one or more diagnostic entities of the output set.

2. The method of claim 1, wherein determining a calibrated probability for each of the determined likelihoods of diagnostic entities being present comprises:
applying one of: temperature scaling, vector scaling matrix scaling, or Dirichlet calibration to the likelihoods output by the ML model to obtain the calibrated probabilities.

3. The method of claim 1, wherein generating the output set comprises applying a conformal prediction technique to the calibrated probabilities.

4. The method of claim 3, wherein applying the conformal prediction technique comprises:
applying a score function to each of the calibrated probabilities to determine respective scores; and
comparing the scores to a threshold score associated with a quantile determined for the ML model;
when the score associated with a diagnostic entity exceeds the threshold score, including the diagnostic entity in the output set.

5. The method of claim 4, wherein the conformal prediction technique is an adaptive prediction set technique.

6. The method of claim 5, wherein the applying the score function comprises:
sorting the calibrated probabilities from most to least likely; and
greedily summing the probabilities.

7. The method of claim 6, wherein the output set is the set of diagnostic entities for which the greedily summed probabilities exceeds the threshold score.

8. The method of claim 3, wherein:
the output set is the set of entities satisfying the conformal prediction technique and/or having a size less than or equal to a maximum set size.

9. The method of claim 8, wherein the maximum set size is three diagnostic entities.

10. The method of claim 1 wherein the at least one trained ML model comprises:
a pre-trained foundational model backbone; and
an adaptation head trained to determine the likelihood of the one or more diagnostic entities being present in one or more tissue samples.

11. The method of claim 1, wherein:
the indications of one or more of the diagnostic entities comprise image data labeled with one or more fields of interest (FOIs) corresponding to a diagnostic entity within the output set.

12. The method of claim 1, wherein:
the indications of the one or more diagnostic entities comprise image data with one or more visual overlays, each overlay indicating the likelihood of one or more diagnostic entities being present.

13. The method of claim 1, wherein the one or more diagnostic entities include: benign nevus, basal cell carcinoma, squamous cell carcinoma, seborrheic keratosis, actinic keratosis, dermatitis, dysplastic nevus, squamous cell carcinoma in situ, verruca vulgaris, benign non-melanocytic lesion, scar, cyst, lichenoid keratosis, melanoma in situ, vascular lesion, invasive melanoma, and/or normal skin.

14. A system for processing of a histopathology image, the system comprising:
at least one computer hardware processor; and
at least one non-transitory computer readable storage medium, storing processor executable instructions, that when executed by the at least one computer hardware processor cause the processor to perform a method comprising:
obtaining the histopathology image, the image containing histopathology data of one or more tissue samples;
processing the histopathology image data using at least one trained machine learning (ML) model to determine a likelihood of one or more diagnostic entities being present in the one or more tissue samples;
determining a calibrated probability for each of the determined likelihoods of diagnostic entities being present;
generating an output set of one or more diagnostic entities, wherein a size of the output set and the diagnostic entities within the output set are determined based on the calibrated probabilities associated with each diagnostic entity; and
causing a user interface to display the output set and indications of the one or more diagnostic entities of the output set.

15. The system of claim 14, wherein determining a calibrated probability for each of the determined likelihoods of diagnostic entities being present comprises:
applying one of: temperature scaling, vector scaling matrix scaling, or Dirichlet calibration to the likelihoods output by the ML model to obtain the calibrated probabilities.

16. The system of claim 14, wherein generating the output set comprises applying a conformal prediction technique to the calibrated probabilities.

17. The system of claim 16, wherein applying the conformal prediction technique comprises:
applying a score function to each of the calibrated probabilities to determine respective scores; and
comparing the scores to a threshold score associated with a quantile determined for the ML model;
when the score associated with a diagnostic entity exceeds the threshold score, including the diagnostic entity in the output set.

18. The system of claim 17, wherein the conformal prediction technique is an adaptive prediction set technique.

19. The system of claim 16, wherein:
the output set is the set of entities satisfying the conformal prediction technique and/or having a size less than or equal to a maximum set size.

20. At least one non-transitory computer readable storage medium, storing processor executable instructions, that when executed by at least one computer hardware processor cause the processor to perform a method comprising:
obtaining a histopathology image, the image containing histopathology data of one or more tissue samples;
processing the histopathology image data using at least one trained machine learning (ML) model to determine a likelihood of one or more diagnostic entities being present in the one or more tissue samples;
determining a calibrated probability for each of the determined likelihoods of diagnostic entities being present;
generating an output set of one or more diagnostic entities, wherein a size of the output set and the diagnostic entities within the output set are determined based on the calibrated probabilities associated with each diagnostic entity; and
causing a user interface to display the output set and indications of the one or more diagnostic entities of the output set.
